(19) United States
(12) Patent Application Publication
(10) Pub. No.: US 2016/0030527 A1
(43) Pub. Date: Feb. 4, 2016

(54) Compositions and Methods for Treatment of Stroke

(71) Applicant: THE TRUSTEES OF THE UNIVERSITY OF PENNSYLVANIA, Philadelphia, PA (US)

(72) Inventors: Katalin Kariko, Rydal, PA (US); Drew Weissman, Wynnewood, PA (US)

(73) Assignee: THE TRUSTEES OF THE UNIVERSITY OF PENNSYLVANIA, Philadelphia, PA (US)

(21) Appl. No.: 14/776,545

(22) PCT Filed: Mar. 13, 2014
(86) PCT No.: PCT/US14/26232

§ 371 Date: Sep. 14, 2015

Related U.S. Application Data
(60) Provisional application No. 61/783,960, filed on Mar. 14, 2013.

(51) Int. Cl. A61K 38/46; C12N 9/16; C12N 9/14; A61K 45/06; C12N 9/22

(52) U.S. Cl. CPC A61K 38/465; A61K 45/06; A61K 38/46; C12N 9/22; C12N 9/14; C12N 9/16; C12Y 301/27005; C12Y 301/21001; C12Y 306/01005; C12Y 301/03005

(57) ABSTRACT

The present invention provides compositions and methods for treating stroke. The invention relates to inhibiting the level and/or activity of cell debris after the onset of stroke. In certain embodiments, the invention provides for the catabolism or inhibition of at least one of extracellular RNA, extracellular DNA, and extracellular ATP.

### CROSS-REFERENCE TO RELATED APPLICATIONS

This application claims priority to U.S. Provisional Application No. 61/783,960 filed on Mar. 14, 2013, the contents of which are incorporated by reference herein in their entirety.

### STATEMENT REGARDING FEDERALLY SPONSORED RESEARCH OR DEVELOPMENT

This invention was made with government support under grant numbers R01NS029331 and R42HL87688, awarded by the National Institutes of Health (NIH). The government has certain rights in the invention.

### BACKGROUND OF THE INVENTION

On average, every 40 seconds, someone in the United States has a stroke and, every 4 minutes, someone dies due to a stroke. Ischemic stroke represents 87% of all strokes and is a major cause of disability accounting for a significant proportion of health service budgets (Roger et al., 2012, Circulation 125(22):e1002). At present, the only available corrective drug treatment for cerebral ischemia is thrombolysis using tissue plasminogen activator (tPA), which has a narrow therapeutic window and helps only 5% of stroke victims (Iadecola and Anrather, 2011, Nat Neurosci 14:1363-1368). Thus, there is an urgent need for safe and effective treatments. For decades, drugs tested in clinical trials were designed to antagonize the body's responses to brain ischemia. Those drugs, including ion channel antagonists, enzyme inhibitors, blockers of Na+/Ca2+-channels, anti-ICAM and anti-integrin antibodies, antioxidants, NMDA and AMPA receptor antagonists, immune suppressors and anti-inflammatory agents demonstrated no benefit for the patients and some were even harmful (FIG. 1) (Norris and Hachinski, 1986, BMJ 292:21-23; De Keyser et al., 1999, Trends Neurosci 22:535-540; Becker, 2002, Curr Med Res Opin 18(Suppl 2):s18-22; Cheng et al., 2004, NeuroRx 1:36-45; Green and Shuaib, 2006, Drug Discovery Today 11:681-693; Green, 2008, Br J Pharmacol 153(Suppl 1):S325-338).

It is believed that suppressing, blocking, or antagonizing molecules and pathways that have been identified as key players in mediating and perpetuating ischemic brain damage does not improve outcome, because the targeted molecules and pathways have dual roles; they are also involved in survival, resolution and regeneration. For example, severe inflammation can be detrimental, but inflammation also mediates the clearance of debris released by injured cells (Roos et al., 2004, Eur J Immunol 34:921-929; Stetson and Medzhitov, 2006, Immunity 24:93-103) and promotes tissue repair (Murray and Wynn, 2011, Nat Rev Immunol 11:723-737). Excessive stimulation of glutamate receptors induces cell death, but their activation also conveys anti-apoptotic signals to survival pathways (Tymianski, 2011, Nat Neurosci 14:1369-1373). IL-1 signaling exacerbates brain injury (Emsley et al., 2005, J Neurol Neurosurg Psychiatry 76:1366-1372), however it also has a protective role and promotes blood vessel remodeling (Alexander et al., 2012, J Clin Invest 122:70-79).

During injury, cells lose their membrane integrity and release their intracellular contents to the extracellular milieu. The escaped molecules interact with components of the extracellular environment, alter their function, and result in pathophysiological changes (Kleine et al., 1995, Am J Physiol 268(5 Pt 1):C1114-25; Kleine et al., 1997, Am J Physiol 273(6 Pt 1):C1925-C1936; Wygrecka et al., 2007, J Biol Chem 282:21671-21682; Xu et al., 2009, Nat Med 15:1318-1321; Nakazawa et al., 2005, Biochem J 385:831-838; Deindl et al., 2009, Indian J Biochem Biophys 46:461-466; Kannemeier et al., 2007, Proc Natl Acad Sci USA 104:6388-6393) Innate immune system pattern recognition receptors also respond to these endogenous ligands generating inflammation that can assist clearance, but can also exacerbate other processes, especially when associated with massive cell injury (e.g. trauma, ischemia, organ transplantation) (Matzinger, 2002, Science 296:301-305). Decreasing the activation of inflammatory pathways in these settings would be desirable, but so far, agents that suppressed, blocked or antagonized pathways activated by endogenous ligands were not beneficial to patients (FIG. 1) (Norris and Hachinski, 1986, BMJ 292:21-23; De Keyser et al., 1999, Trends Neurosci 22:535-540; Becker, 2002, Curr Med Res Opin 18(Suppl 2):s18-22; Cheng et al., 2004, NeuroRx 1:36-45; Green, 2008, Br J Pharmacol 153(Suppl 1):S325-338). Better understanding of both the molecular mechanisms of the escaped intracellular components and the extracellular environment, as well as the signaling mechanisms of pattern recognition receptors provided new targets and renewed interest in the treatment of stroke. Some results suggest that blocking Toll-like receptor (TLR) 4, the receptor for lipopolysaccharide and numerous endogenous ligands, might be beneficial, as others and we have demonstrated. TLR4-deficient mice subjected to intracerebral hemorrhage (ICH) (Sansing et al., 2011, Ann Neurol 70:646-656) or middle cerebral artery occlusion (MCAO) (Caso et al., 2007, Circulation 115:1599-1608) had better outcomes than wild-type mice, although other results demonstrated no effect of interrupting TLR signaling (MyD88 null, IRF3 null, IRF7 null, TRIF mutant mice) on the infarct size following MCAO (Marsh et al., 2009, J Neurosci 29(31):9839-9849; Famakin et al., 2011, Brain Res 1388:148-156; Stevens et al., 2011, J Neurosci 31:8456-8463).

The cellular contents released during cell death including, ATP, RNA, DNA, nucleosomes, histones, and HMGB1 are well documented to induce sterile inflammation (Deindl et al., 2009, Indian J Biochem Biophys 46:461-466; Zhang et al., 2010, Nature 464:104-107; Kim et al., 2006, J Neurosci. 26:6413-6421; Scaffidi et al., 2002, Nature 418:191-195; Xu et al., 2011, J Immunol 187(5):2626-2631) and activate multiple other systems leading to pathological coagulation (Wygrecka et al., 2007, J Biol Chem 282:21671-21682; Xu et al., 2009, Nat Med 15:1318-1321; Nakazawa et al., 2005, Biochem J 385:831-838; Kannemeier et al., 2007, Proc Natl Acad Sci USA 104:6388-6393; Semeraro et al., 2011, Blood 118:1952-1961), complement activation (Zhang et al., 2010, Molecular Immunology 47:2225; Kanse et al., 2012, J Immunol 188(6):2858-2865), and endothelial cell dysfunction that leads to vascular leakiness and edema (Thompson et al., 2004, J Exp Med 200:1395-1405; Fischer et al., 2007, Blood 110:2457-2465; Fischer et al., 2009, FASEB J 23(7):2100-2109; Kleine et al., 1995, Am J Physiol 268(5 Pt 1):C1114-25; Kleine et al., 1997, Am J Physiol 273(6 Pt 1):C1925-C1936).

Extracellular RNA is taken up and recognized by pattern recognition receptors such as TLR3, TLR7, TLR8, RIG-I, MDA5, DDX, NOD2, NALP3, IFIT5, PKR and 2′-5′-oligoadenylate synthetase (OAS) resulting in immune activation, including proinflammatory cytokine release (Takeuchi and Akira, 2010, Cell 140:805-820; Zhang et al., 2011, Immunity 34:866-878). Extracellular RNA also promotes pathologic thrombosis by several different pathways. RNA binds and activates Factor VII-activating protease, which is a potent activator of coagulation Factor VII (Nakazawa et al., 2005, Biochem J 385:831-838). RNA also serves as a cofactor for the Factor XII/XI-induced contact activation/amplification of blood coagulation (Kannemeier et al., 2007, Proc Natl Acad Sci USA 104:6388-6393). Furthermore, extracellular RNA binds to plasminogen activator inhibitor-1 (PAI-1) and stabilizes the active conformational state of PAI-1, which binds and inactivates thrombolytic tPA and uPA (Wygrecka et al., 2007, J Biol Chem 282:21671-21682). Additionally, extracellular RNA increases permeability of microvascular endothelial cells through a VEGF-mediated mechanism, impairs the blood-brain-barrier and contributes to vasogenic edema (Deindl et al., 2009, Indian J Biochem Biophys 46:461-466; Fischer et al., 2007, Blood 110:2457-2465; Walberer et al., 2009, Curr Neurovasc Res 6:12-19).

Extracellular ATP activates the NALP3 inflammasome resulting in caspase activation that leads to IL-1β and IL-18 release (Mariathasan et al., 2006, Nature 440:228-232). ATP also induces neurodegeneration by activating ionotropic purinergic receptor P2X7 (Domercq et al., 2010, Glia 58:730-740) and increases vascular leakage and transendothelial migration of lymphoid cells (Yegutkin, 2008, Biochim Biophys Acta 1783:673-694; Yegutkin et al., 2011, Angiogenesis 14:503-513). Extracellular ATP acting through metabotropic P2Y2 receptor increases IL-8 production (Kukulski et al., 2011, J Immunol 187:644-653).

Extracellular DNA activates endosomal TLR9 and cytoplasmic DAI and DDx41 receptors resulting in proinflammatory cytokine release (Zhang et al., 2010, Nature 464:104-107; Leadbetter et al., 2002, Nature 416:603-607; Muruve et al., 2008, Nature 452:103-107; Zhang et al., 2011, Nat Immunol 12:959-965). It also activates ASC of the inflammasome resulting in caspase-mediated cleavage of pro-IL-1β (Muruve et al., 2008, Nature 452:103-107). DNA can also promote coagulation by binding to and increasing the half-life of PAI-1 (Wygrecka et al., 2007, J Biol Chem 282:21671-21682) or by binding to coagulation Factors XII and XI and augmenting coagulation (Kannemeier et al., 2007, Proc Natl Acad Sci USA 104:6388-6393).

Histones are normally contained within the nucleus, but, during cell damage, they are released to the extracellular milieu as part of the nucleosome. Histones increase cell permeability forming channels in cell membranes that leads to cellular swelling (Kleine et al., 1995, Am J Physiol 268(5 Pt 1):C1114-25; Kleine et al., 1997, Am J Physiol 273(6 Pt 1):C1925-C1936). Histones also enhance the DNA-activated TLR9 signaling cascade (Huang et al., 2011, Hepatology 54:999-1008) and can directly activate TLR2 and TLR4, leading to proinflammatory cytokine production and tissue injury (Xu et al., 2011, J Immunol 187(5):2626-2631).

HMGB1 is a nuclear protein that is also present in the plasma at low levels, which leaks out of damaged cells and induces inflammation (Scaffidi et al., 2002, Nature 418:191-195). Extracellular HMGB1 interacts with receptors, including those for advanced glycation endproducts (RAGEs) (Muhammad et al., 2008, J Neurosci 28:12023-12031) as well as TLR2 and TLR4 (Park et al., 2004, J Biol Chem 279:7370-7377; Yang et al., 2010, J Cereb Blood Flow Metab 30(2):243-254; Maroso et al., 2010, Nat Med 16:413-419). HMGB1 also binds to nucleic acids and promote activation of TLR3, TLR7 and TLR9 by their respective ligands (Tian et al., 2007, Nat Immunol 8:487-496; Yanai et al., 2009, Nature 462:99-103).

Peroxiredoxins are intracellular neuroprotective enzymes with antioxidant properties. However, when peroxiredoxins are released from necrotic brain cells, they lose their enzymatic activity and become danger signals acting on TLR2 and TLR4 (Shichita et al., 2012, Nat Med 18:911-917; Garcia-Bonilla and Iadecola, 2012, Nat Med 18:858-859).

It is well documented that various types of cell debris released from injured cells can either directly cause or exacerbate multiple diseases, including ischemic stroke (Muhammad et al., 2008, J Neurosci 28:12023-12031; Shichita et al., 2012, Nat Med 18:911-917; Garcia-Bonilla and Iadecola, 2012, Nat Med 18:858-859), intracerebral hemorrhage (Guo et al., 2012, Transl Stroke Res 3:130-137), sepsis (Xu et al., 2009, Nat Med 15:1318-1321), systemic inflammatory response syndrome (Zhang et al., 2010, Nature 464:104-107), age-related macular degeneration (Ambati and Fowler, 2012, Neuron 75:26-39), rheumatoid arthritis (Hajizadeh et al., 2003, Arthritis Res Ther 5:R234-240; Brentano et al., 2005, Arthritis Rheum 52:2656-2665), lupus (Decker et al., 2005, J Immunol 174:3326-3334; Barrat et al., 2005, J Exp Med 202:1131-1139; Leadbetter et al., 2002, Nature 416:603-607), chronic renal failure (Kocic et al., 2010, Renal Failure 32:486-492), ischemia reperfusion injuries of brain, heart, liver, kidney and intestine (Wu et al., 2007, J Clin Invest 117:2847-2859; Arumugam et al., 2009, Shock 32:4-16; Chen et al., 2014, J Am Heart Assoc, 3: e000683). Antagonizing extracellular HMGB1 with neutralizing antibodies reduced inflammation and ameliorated ischemic brain damage in a mouse model of MCAO (Muhammad et al., 2008, J Neurosci 28:12023-12031). RNase1 delivered intravenously reduced brain edema and the size of the infarct in a rat MCAO model (Walberer et al., 2009, Curr Neurovasc Res 6:12-19). Antibodies targeted to extracellular histones prevented death of animals with bacterial sepsis (Xu et al., 2009, Nat Med 15:1318-1321). ATP is released from dying cells and has pleotropic effects through both nucleotide receptors and inflammasomes. Although, ATP catabolizing enzymes ENTPD1 (also known as apyrase, CD39) and NT5E (5′-nucleotidase, CD73) have not been tested on animal models of stroke, results obtained with other disease models demonstrate important supportive data. When lungs were instilled with the soluble form of ENTPD1 (catabolizes ATP to AMP) prior to implantation, lung reperfusion injury was reduced (Sugimoto et al., 2009, J Thorac Cardiovasc Surg 138:752-759). Soluble ENTPD1 also inhibited leukocyte infiltration and neointimal formation (Drosopoulos et al., 2010, Thromb Haemost 103:426-434), hypothermia-induced platelet aggregation and thrombosis formation in mouse injury models (Straub et al., 2011, Arterioscler Thromb Vasc Biol 31:1607-1616). It has been shown that hypoxia drastically impairs both ENTPD1 and NT5E activity leading to increased endothelial cell permeability and edema (Yegutkin et al., 2011, Angiogenesis 14:503-513), which is a very serious complication of cerebral ischemia. Administration of soluble NT5E completely inhibited hypoxia-induced vascular leakage (Thompson et al., 2004, J Exp Med 200:1395-1405) and immune cell infiltration (Reutershan et al., 2009, FASEB J 23:473-482). Interestingly, treatment with IFN-β could also block vascular leakage by inducing the expression and activity of NT5E on endothelial cells (Kiss et al., 2007, Eur J Immunol 37:3334-3338). Thus, IFN-β-induced NT5E might mediate the major reduction of infarct volumes (Marsh et al., 2009, J Neurosci 29(31):9839-9849; Liu et al., 2002, Neurosci Lett 327:146-148; Veldhuis et al., 2003, J Cereb Blood Flow Metab 23:1029-1039) and enhance neuronal survival (da Silva and Jones, 2012, J Virol 86:1670-1682; Dann et al., 2012, Nat Neurosci 15:98-106) of IFN-β-treated animals with stroke. Neutralizing antibodies to peroxiredoxins reduced infarct volume and ameliorated motor deficits in mice with MCAO (Shichita et al., 2012, Nat Med 18:911-917; Garcia-Bonilla and Iadecola, 2012, Nat Med 18:858-859).

Necrotic cell-released debris is well recognized as a major instigator of inflammation, coagulation, complement activation, and endothelial cell dysfunction that exacerbates disease pathogenesis. As detailed above, approaches to eliminate portions of the debris by delivering catabolizing enzymes in their protein form or neutralize them with antibodies have been successful (Xu et al., 2009, Nat Med 15:1318-1321; Thompson et al., 2004, J Exp Med 200:1395-1405; Walberer et al., 2009, Curr Neurovasc Res 6:12-19; Muhammad et al., 2008, J Neurosci 28:12023-12031; Shichita et al., 2012, Nat Med 18:911-917; Garcia-Bonilla and Iadecola, 2012, Nat Med 18:858-859; Sugimoto et al., 2009, J Thorac Cardiovasc Surg 138:752-759; Drosopoulos et al., 2010, Thromb Haemost 103:426-434; Straub et al., 2011, Arterioscler Thromb Vasc Biol 31:1607-1616; Reutershan et al., 2009, FASEB J 23:473-482). An alternative approach that removed extracellular nucleic acids from the blood by binding them to a special polymer also demonstrated promising results (Lee et al., 2011, Proc Natl Acad Sci 108(34):14055-14060).

However, despite the prior approaches, there is a need in the art for compositions and methods to effectively eliminate cell debris for treatment of stroke. The present invention satisfies this unmet need.

### SUMMARY OF THE INVENTION

The present invention provides a composition for treating stroke. In one aspect, the composition comprises at least one isolated nucleic acid encoding at least one cell debris inhibitor. In one embodiment, the at least one cell debris inhibitor is a catabolizing enzyme. In one embodiment, the at least one cell debris inhibitor is at least one selected from the group consisting of an RNase, a DNase, an ENTPD, and NT5E.

In one embodiment, the at least one isolated nucleic acid comprises in vitro transcribed RNA. In one embodiment, the at least one isolated nucleic acid comprises nucleoside-modified RNA. In one embodiment, the at least one isolated nucleic acid comprises pseudouridine.

In one embodiment, the composition comprises an isolated nucleic acid encoding an RNase, an isolated nucleic acid encoding a DNase, an isolated nucleic acid encoding an ENTPD, and an isolated nucleic acid encoding NT5E.

In one embodiment, the composition further comprises an isolated nucleic acid encoding an inhibitor of HMGB1. In one embodiment, the composition further comprises an isolated nucleic acid encoding an inhibitor of peroxiredoxins. In one embodiment, the composition further comprises a cell debris-inhibiting or -catabolizing peptide.

In one aspect, the composition for treating stroke comprises at least one cell debris-inhibiting or -catabolizing peptide. In one embodiment, the at least one cell debris-inhibiting or -catabolizing peptide is a catabolizing enzyme. In one embodiment, the at least one cell debris-inhibiting or -catabolizing peptide is at least one selected from the group consisting of an RNase, a DNase, an ENTPD, and NT5E. In one embodiment, the composition comprises an RNase, a DNase, an ENTPD, and NT5E.

In one embodiment, the composition further comprises an inhibitor of HMGB1. In one embodiment, the composition further comprises an inhibitor of peroxiredoxins.

The present invention provides a method of treating stroke comprising administering to a subject an effective amount of a composition comprising at least one isolated nucleic acid encoding at least one cell debris inhibitor. In one embodiment, the at least one cell debris inhibitor is a catabolizing enzyme. In one embodiment, the at least one cell debris inhibitor is at least one selected from the group consisting of an RNase, a DNase, an ENTPD, and NT5E.

In one embodiment, the at least one isolated nucleic acid comprises in vitro transcribed RNA. In one embodiment, the at least one isolated nucleic acid comprises nucleoside-modified RNA. In one embodiment, the at least one isolated nucleic acid comprises pseudouridine.

In one embodiment, the composition comprises an isolated nucleic acid encoding at least one selected from the group consisting of an RNase, an isolated nucleic acid encoding a DNase, an isolated nucleic acid encoding an ENTPD, and an isolated nucleic acid encoding NT5E.

In one embodiment, the composition further comprises an isolated nucleic acid encoding an inhibitor of HMGB1. In one embodiment, the composition further comprises an isolated nucleic acid encoding an inhibitor of peroxiredoxins. In one embodiment, the composition further comprises a cell debris-inhibiting or -catabolizing peptide.

In one embodiment, the composition is administered by a delivery method selected from the group consisting of intravenous delivery, intranasal delivery, and intracerebroventricular delivery.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a diagram of the mechanisms involved in the “brain ischemic cascade” with compounds tested in clinical trials as therapeutics to antagonize physiological responses to ischemic insult.

FIG. 2, comprising FIG. 2A through FIG. 2E, is a set of graphs depicting the results of experiments demonstrating the increased amounts of extracellular nucleosomes, RNA, DNA, ATP and IL-6 are circulating in the plasma of mice following MCAO. Mice (n=3) were subjected to 30 mins of transient MCAO. Their plasma was analyzed at 4 and 24 h after reperfusion. Sham-operated animals (n=3) were used for comparison. Nucleosomes (FIG. 2A) were measured using the Cell Death Detection ELISA. Extracellular RNA (FIG. 2B) and DNA (FIG. 2C) were determined by RT-qPCR and qPCR, respectively. Levels of plasma ATP (FIG. 2D) were measured in bioluminescence assays. IL-6 was determined using ELISA (FIG. 2E).

FIG. 3 is a set of graphs and images depicting the results of experiments demonstrating that inflammatory ligands and cytokines down-regulate RNase1 mRNA in dendritic cells and PBMCs. Monocyte-derived dendritic cells (MDDC) derived by IL-4 and GM-CSF treatment of human monocytes and human PBMCs were stimulated for 20 h with the following ligands: GM-CSF (50 ng/ml), IL-4 (100 ng/ml), IFN-α (1000 U/ml), LPS (1 ng/ml), IL-1β (50 ng/ml), IL-12 (50 ng/ml), polyI:C (50 μg/ml), R-848 (1 μg/ml), TNF-α (2 ng/ml), ODN (5 μM), LTA (2 μg/ml). Isolated total RNA was analyzed by Northern blot using RNase1 and GAPDH probes. Autoradiograms of the blots (upper panel) and their corresponding densitometric profile (lower panel) normalized for RNA loading and values of the untreated samples are shown.

FIG. 4, comprising FIG. 4A and FIG. 4B, are a set of graphs depicting the results of experiments demonstrating that proinflammatory cytokine treatments reduce expression of RNase1, ENTPD1 and NT5E by human umbilical vein and human microvascular endothelial cells (HUVECs and HMECs). (FIG. 4A) Data from IL-1β treated HUVECs. (FIG. 4B) Results for NT5E and RNase1 expression obtained from 4 and 3 donors, respectively, are shown. Adapted from GEO DataSets as indicated.

FIG. 5 is a graph depicting the results of experiments demonstrating that administration of EPO mRNA increases serum EPO levels in mice. TransIT-complexed mRNA (0.1 μg) coding for murine EPO containing pseudouridine (EPO Ψ-mRNA) or containing uridine (EPO U-mRNA) or coding for firefly luciferase and containing pseudouridine (luc Ψ-mRNA) or with 3 μg of recombinant murine EPO (rmEPO) protein were injected i.p. Serum EPO levels were measured by ELISA at the indicated time points. Five animals per condition were analyzed

FIG. 6 is a graph depicting the results of experiments demonstrating that the administration of RNase1 mRNA significantly increases RNase activity in the plasma of mice. TransIT-complexed, pseudouridine-containing mRNA (5 μg) coding for murine RNase1 or beta-lactamase (control mRNA) were injected i.p. At the indicated time points, RNase activity in the plasma was measured on a fluorometer using an adapted assay, which utilizes dual-labeled 6-FAM (fluorescent) and BHQ-1 (quencher) RNA substrate (RNaseAlert kit, Ambion). Three animals per condition were analyzed.

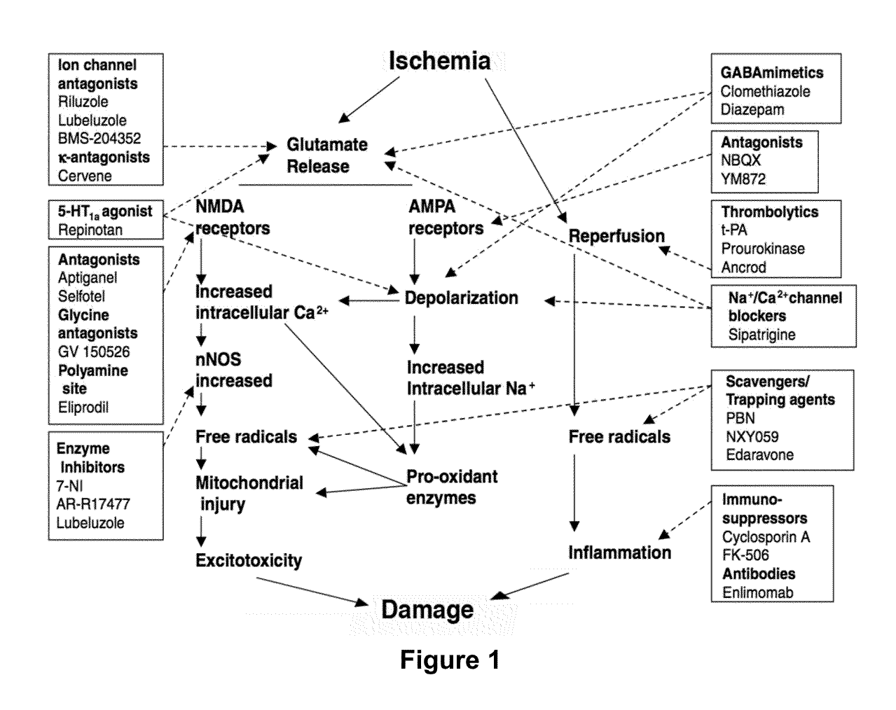

### DETAILED DESCRIPTION

The present invention generally relates to compositions and methods to treat stroke, including, for example, cerebral ischemic stroke. In certain embodiments, the invention is based upon the focused elimination of intracellular molecules that are released from injured cells. The elimination of necrotic cell debris leads to improved recovery after the onset of stroke.

In one embodiment, the invention provides a composition comprising an inhibitor of released cell debris. For example, in certain embodiments, the composition provides for the catabolism and/or inhibition of released cell debris. In one embodiment, the composition provides for expression of a catabolizing enzyme that eliminates cell debris.

In one embodiment the composition eliminates at least one of extracellular RNA, extracellular DNA, and extracellular ATP that is released after stroke. The present invention is partly based upon the finding that RNA, DNA, and ATP are among the cell debris released after stroke and lead to inflammation and secondary cell death. Further, the present invention is partly based upon the finding that during injury and inflammatory conditions when more debris is circulating, the expression and/or activity of the catabolizing enzymes that eliminate extracellular RNA, extracellular DNA, and extracellular ATP are decreased or inhibited. In one embodiment, the composition of the invention provides for enhanced expression of a ribonuclease (RNase) to eliminate extracellular RNA. In one embodiment, the invention provides for enhanced expression of a deoxyribonuclease (DNase) to eliminate extracellular DNA. In one embodiment, the invention provides for enhanced expression of an Ectonucleoside triphosphate diphosphohydrolase (ENTPD) and/or NT5E to eliminate extracellular ATP. In certain embodiments, the composition provides for inhibition of additional cell debris that is released after stroke. For example, in certain embodiments, the composition comprises an inhibitor of the expression and/or activity of histones, HMGB1, or peroxiredoxins.

In certain embodiments, the composition of the invention eliminates extracellular RNA, extracellular DNA, and extracellular ATP that is released after stroke. In some instances, targeting more than one type of cell debris for elimination is preferred considering the pleiotropic effects of those molecules. For example, in certain instances extracellular RNA, DNA and ATP potentiate each other's toxicities, and thus simultaneous elimination of debris has an added or synergistic benefit.

In one embodiment, the composition of the invention comprises an isolated nucleic acid that eliminates at least one of extracellular RNA, extracellular DNA, and extracellular ATP that is released after stroke. In certain embodiments, the composition comprises an isolated nucleic acid encoding for at least one of an RNase, a DNase, ENTPD, and NT5E.

In one embodiment, the composition of the invention comprises in vitro transcribed (IVT) RNA that eliminates at least one of extracellular RNA, extracellular DNA, and extracellular ATP that is released after stroke. In certain embodiments, the composition comprises IVT RNA encoding for at least one of an RNase, a DNase, ENTPD, and NT5E.

In one embodiment, the composition of the invention comprises nucleoside-modified mRNA that eliminates at least one of extracellular RNA, extracellular DNA, and extracellular ATP that is released after stroke. In certain embodiments, the composition comprises nucleoside-modified mRNA encoding for at least one of an RNase, a DNase, ENTPD, and NT5E.

In certain embodiments, the composition provides transient and scalable expression of at least one of an RNase, a DNase, ENTPD, and NT5E. For example, use of IVT RNA provides transient and scalable expression which mitigates the risks of long-term enhanced expression. In some embodiments, the composition of the invention provides for stable, safe, and efficient expression of at least one of an RNase, a DNase, ENTPD, and NT5E. For example, use of nucleoside-modified mRNA makes the nucleic acid more stable, non-immunogenic, and highly translatable.

In one embodiment, the composition of the invention comprises an isolated peptide that eliminates at least one of extracellular RNA, extracellular DNA, and extracellular ATP that is released after stroke. In certain embodiments, the composition comprises at least one of an RNase, a DNase, ENTPD, and NT5E. In one embodiment, the composition of the invention comprises an isolated nucleic acid and an isolated peptide which eliminates at least one of extracellular RNA, extracellular DNA, and extracellular ATP that is released after stroke. In certain embodiments, delivery of both an isolated peptide and an isolated nucleic acid allows for an initial bolus of an active peptide along with a delayed and more sustained delivery of the peptide as encoded by the nucleic acid.

In one embodiment, the present invention provides a method for treating a subject who is having or who has had a stroke comprising administering to the subject an effective amount of a composition that eliminates at least one of extracellular RNA, extracellular DNA, and extracellular ATP that is released after stroke. In certain embodiments, the method of the invention allows for sustained presence of the inhibitors of cell debris, described herein, for at least several days post the onset of stroke. The invention includes treatment of cerebral ischemia, subarachnoid hemorrhage, and intracerebral hemorrhage.

In some embodiments, the method comprises administering to the subject a composition comprising nucleoside-modified mRNA encoding at least one of an RNase, a DNase, ENTPD, and NT5E. In one embodiment, the method comprises administering to the subject a composition comprising nucleoside-modified mRNA encoding an RNase, a DNase, ENTPD, and NT5E.

In one embodiment, the method of the invention comprises systemic administration of the subject, including for example enteral or parenteral administration. In certain embodiments, the method comprises intranasal delivery of the composition. In another embodiment, the method comprises intravenous delivery of the composition. In some embodiments, the method comprises intracerebroventricular delivery of the composition.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody, which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned) or other technology, which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term, “cell debris” as used herein refers cellular components that are released into the extracellular space during or after the death of the cell. In certain instances, cell debris comprises cellular components that are typically intracellular. Exemplary cell debris includes, but is not limited to, RNA, DNA, ATP, histones, HMGB1, and peroxiredoxins.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleosides (nucleobase bound to ribose or deoxyribose sugar via N-glycosidic linkage) are used. “A” refers to adenosine, “C” refers to cytidine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In certain instances, the polynucleotide or nucleic acid of the invention is a “nucleoside-modified nucleic acid,” which refers to a nucleic acid comprising at least one modified nucleoside. A “modified nucleoside” refers to a nucleoside with a modification. For example, nearly one hundred different nucleoside modifications have been identified in RNA (Rozenski, et al., 1999, The RNA Modification Database: 1999 update. Nucl Acids Res 27: 196-197).

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence. For example, the promoter that is recognized by bacteriophage RNA polymerase and is used to generate the mRNA by in vitro transcription.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “stroke” as used herein refers to a condition in which blood flow to a region within the brain is disrupted. Stroke includes, but is not limited to, blockage, hemorrhage, cerebral ischemia, intracranial hemorrhage, and intracerebral hemorrhage, subarachnoid hemorrhage. “Cerebral ischemia” or “brain ischemia” refers to conditions in which blood supply within a region of the brain is interrupted, and can be caused by a variety of reasons including, but not limited to, a thrombosis, embolism, systemic hypoperfusion, and venous thrombosis.

The term “therapeutic” as used herein means a treatment and/or prophylaxis. A therapeutic effect is obtained by suppression, diminution, remission, or eradication of at least one sign or symptom of a disease or disorder state.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compositions

The present invention provides a composition comprising at least one inhibitor of cell debris. As used herein, “cell debris” refers to intracellular components that are released from dead or dying cells. As described herein, in certain instances stroke causes the release of cell debris into brain tissue and surrounding areas, as well as into the circulation, which in turn results in inflammation, coagulation, endothelial cell dysfunction, secondary cell death, and/or increased brain damage. Examples of cell debris include, but are not limited to, extracellular RNA, extracellular DNA, extracellular ATP, histones, HMGB1, and peroxiredoxins.

In some embodiments, the inhibitor of cell debris is an isolated nucleic acid encoding a catabolizing enzyme that eliminates cell debris. In one embodiment, the inhibitor of cell debris is a catabolizing enzyme that eliminates cell debris. For example, a catabolizing enzyme of the invention includes, but is not limited to, an RNase, a DNase, ENTPD, and NT5E.

RNases are a family of enzymes that catalyze the degradation of RNA.

The present invention encompasses any member of the RNase family, including but not limited to, RNase A, RNase1, RNase 3, RNase5, RNase7, RNase8, Dicer, RNase H, RNase I, RNase III, RNase L, RNase P, RNase P, RNase PhyM, RNase T1, RNase T2, RNase U2, RNase U2, RNase V1, RNase V, RNase PH, RNase II, RNase R, RNase D, RNase T, polynucleotide phosphorylase (PNPase), oligoribonuclease, exoribonuclease I, exoribonuclease II and viral-encoded RNases, including but not limited to Flaviviridae RNases, the extracellular RNases of Classic Swine Fever Virus and Bovine Viral Diarrhea Virus.

DNases are a family of enzymes that catalyze the degradation of DNA. The present invention encompasses any member of the DNase family, including but not limited to, DNase I, DNase II, and DNase II beta.

ENTPDs (also known as apyrases) are a family of ecto-nucleosidases that hydrolyze 5′-triphsophates, including ATP. The present invention encompasses any member of the ENTPD family, including but not limited to ENTPD1, ENTPD2, ENTPD3, ENTPD4, ENTPD5, ENTPD6, ENTPD7, and ENTPD8.

NT5E (also known as 5′-NT, ecto-5′-nucleotidase, and CD73) is an enzyme that catalyzes AMP to adenosine.

In certain embodiments, the catabolizing enzymes, or isolated nucleic acids encoding the catabolizing enzymes, are modified to alter the localization, activity, stability, and the like, of the enzyme. For example, in some embodiments, the catabolizing enzyme is modified to be secretable. Endogenous NT5E, and at least some ENTPDs, are membrane bound enzymes. In certain embodiments, the isolated nucleic acid of the invention is modified to encode a secretable form of NT5E and ENTPD. For example, in one embodiment, the encoded enzyme is modified to express a secretion sequence. In one embodiment, the encoded enzyme is modified to remove a transmembrane sequence.

In one embodiment, the inhibitor of cell debris is an in vitro synthesized nucleic acid encoding a peptide that inhibits the expression and/or activity of cell debris. In another embodiment, the inhibitor of cell debris is a peptide that inhibits the expression and/or activity of cell debris. For example, in certain embodiments, the composition of the invention provides for the expression of peptide sequences, antibodies, and the like that inhibit the activity of cell debris including, but not limited to, extracellular RNA, extracellular DNA, extracellular ATP, histones, HMGB1, and peroxiredoxins. In certain embodiments, the composition of the invention comprises a Box A fragment, or isolated nucleic acid encoding the same, to neutralize and/or inhibit the activity of released HMGB1. In another embodiment, the composition of the invention comprises an antibody or antibody fragment, or in vitro synthesized nucleic acid encoding the same, to inhibit the activity of peroxiredoxins.

As used herein, a “cell debris-inhibiting or -catabolizing peptide” therefore encompasses a catabolizing enzyme, as described herein, as well as peptides, proteins, antibodies, and the like which inhibit the activity of cell debris. Further, a “cell debris inhibitor” or “inhibitor of cell debris” encompasses a cell debris-inhibiting or -catabolizing peptide described herein as well as in vitro synthesized nucleic acids encoding a cell debris-inhibiting or -catabolizing peptide. A cell debris inhibitor further encompasses nucleic acids, siRNA, antisense, aptamers, ribozymes, small molecules, and the like which inhibit the activity of cell debris described herein.

### Nucleic Acids

In one embodiment, the invention includes an in vitro-transcribed nucleic acid. In one embodiment, the in vitro-transcribed nucleic acid is an inhibitor of cell debris. In another embodiment, the in vitro-transcribed nucleic acid encodes an inhibitor of cell debris. In one embodiment, the in vitro-transcribed nucleic acid encodes a catabolizing enzyme that catabolizes cell debris.

In one embodiment, the invention includes an in vitro-transcribed nucleic acid encoding an RNase. In one embodiment, the invention includes an in vitro-transcribed nucleic acid encoding a DNase. In one embodiment, the invention includes an in vitro-transcribed nucleic acid encoding an ENTPD. In one embodiment, the invention includes an in vitro-transcribed nucleic acid encoding NT5E. In one embodiment, the invention includes an in vitro-transcribed nucleic acid sequence encoding an RNase, a DNase, an ENTPD, and NT5E. In one embodiment, the composition of the invention comprises an in vitro-transcribed nucleic acid encoding an RNase, an in vitro-transcribed nucleic acid encoding a DNase, an in vitro-transcribed nucleic acid encoding an ENTPD, and an in vitro-transcribed nucleic acid encoding NT5E.

The nucleotide sequences encoding a cell debris-inhibiting or -catabolizing peptide described herein can alternatively comprise sequence variations with respect to the original nucleotide sequences, for example, substitutions, insertions and/or deletions of one or more nucleotides, with the condition that the resulting polynucleotide encodes a polypeptide according to the invention. Therefore, the scope of the present invention includes nucleotide sequences that are substantially homologous to the nucleotide sequences recited herein and encode a cell debris-inhibiting or -catabolizing peptide.

As used herein, a nucleotide sequence is “substantially homologous” to any of the nucleotide sequences described herein when its nucleotide sequence has a degree of identity with respect to the nucleotide sequence of at least 60%, advantageously of at least 70%, preferably of at least 85%, and more preferably of at least 95%. A nucleotide sequence that is substantially homologous to a nucleotide sequence encoding a cell debris-inhibiting or -catabolizing peptide can typically be isolated from a producer organism of the polypeptide of the invention based on the information contained in the nucleotide sequence by means of introducing conservative or non-conservative substitutions, for example. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art. The identity between two amino acid sequences is preferably determined by using the BLASTN algorithm (BLAST Manual, Altschul, S., et al., NCBI NLM NIH Bethesda, Md. 20894, Altschul, S., et al., J. Mol. Biol. 215: 403-410 (1990)).

In another aspect, the invention relates to a construct, comprising a nucleotide sequence encoding a cell debris-inhibiting or -catabolizing peptide or a derivative thereof. In a particular embodiment, the construct is operatively bound to a transcription control element. In another particular embodiment, the construct is operatively bound to a translational control element. The construct can incorporate an operatively bound regulatory sequence for the expression of the nucleotide sequence of the invention, thus forming an expression cassette.

Vectors

The nucleic acid sequences coding for the inhibitor molecules of the invention can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically.

The present invention also provides vectors in which a nucleic acid of the present invention is incorporated. In brief summary, the expression of natural or synthetic nucleic acids encoding a cell debris-inhibiting or -catabolizing peptide is typically achieved by operably linking a nucleic acid encoding a cell debris-inhibiting or -catabolizing peptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The expression constructs of the present invention may also be used for gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, sequencing vectors and vectors optimized for in vitro transcription.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/RNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the mRNA sequence in the host cell, a variety of assays may be performed. Such assays include, for example, “molecular biological” assays well known to those of skill in the art, such as Northern blotting and RT-PCR; “biochemical” assays, such as detecting the presence or absence of a particular peptide, e.g., by immunological means (ELISAs and Western blots) or by assays described herein to identify agents falling within the scope of the invention.

In Vitro Transcribed RNA

In one embodiment, the composition of the invention comprises in vitro transcribed (IVT) RNA encoding a cell debris-inhibiting or -catabolizing peptide. In one embodiment, an IVT RNA can be introduced to a cell as a form of transient transfection. The RNA is produced by in vitro transcription using a plasmid DNA template generated synthetically. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is a cell debris catabolizing or inhibiting peptide of the present invention.

In one embodiment, the DNA to be used for PCR contains an open reading frame. The DNA can be from a naturally occurring DNA sequence from the genome of an organism. In one embodiment, the DNA is a full length gene of interest of a portion of a gene. The gene can include some or all of the 5′ and/or 3′ untranslated regions (UTRs). The gene can include exons and introns. In one embodiment, the DNA to be used for PCR is a human gene. In another embodiment, the DNA to be used for PCR is a human gene including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

Genes that can be used as sources of DNA for PCR include genes that encode polypeptides that provide a therapeutic or prophylactic effect to an organism or that can be used to diagnose a disease or disorder in an organism. Preferred genes are genes which are useful for a short term treatment, or where there are safety concerns regarding dosage or the expressed gene. For example, for treatment of cancer, autoimmune disorders, parasitic, viral, bacterial, fungal or other infections, the transgene(s) to be expressed may encode a polypeptide that functions as a ligand or receptor for cells of the immune system, or can function to stimulate or inhibit the immune system of an organism. In some embodiments, t is not desirable to have prolonged ongoing stimulation of the immune system, nor necessary to produce changes which last after successful treatment, since this may then elicit a new problem. For treatment of an autoimmune disorder, it may be desirable to inhibit or suppress the immune system during a flare-up, but not long term, which could result in the patient becoming overly sensitive to an infection.

In various embodiments, plasmid is used to generate a template for in vitro transcription of mRNA which is used for transfection.

Chemical structures with the ability to promote stability and/or translation efficiency may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 RNA polymerase promoter, as described elsewhere herein. Other useful promoters include, but are not limited to, T3 and SP6 RNA polymerase promoters. Consensus nucleotide sequences for T7, T3 and SP6 promoters are known in the art.

In a preferred embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is effective in eukaryotic transfection when it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which can be ameliorated through the use of recombination incompetent bacterial cells for plasmid propagation.

Poly(A) tails of RNAs can be further extended following in vitro transcription with the use of a poly(A) polymerase, such as E. coli polyA polymerase (E-PAP) or yeast polyA polymerase. In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to mRNA molecules. In a preferred embodiment, RNAs produced by the methods to include a 5′ cap1 structure. Such cap1 structure can be generated using Vaccinia capping enzyme and 2′-O-methyltransferase enzymes (CellScript, Madison, Wis.). Alternatively, 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001). In certain embodiments RNA of the invention is introduced to a cell with a method comprising the use of TransIT®-mRNA transfection Kit (Mirus, Madison Wis.), which, in some instances, provides high efficiency, low toxicity, transfection.

Nucleoside-Modified RNA

In one embodiment, the composition of the present invention comprises a nucleoside-modified nucleic acid encoding a cell debris-inhibiting or -catabolizing peptide described herein. For example, in one embodiment, the composition comprises a nucleoside-modified RNA. In one embodiment, the composition comprises a nucleoside-modified mRNA. Nucleoside-modified mRNA have particular advantages over non-modified mRNA, including for example, increased stability, low immunogenicity, and enhanced translation. Nucleoside-modified mRNA useful in the present invention is further described in U.S. Pat. No. 8,278,036, which is incorporated by reference herein in its entirety.

In certain embodiments, nucleoside-modified mRNA does not activate any pathophysiologic pathways, translate very efficiently and almost immediately following delivery, and serve as templates for continuous protein production in vivo lasting for several days (Karikó et al., 2008, Mol Ther 16:1833-1840; Karikó et al., 2012, Mol Ther 20:948-953). The amount of mRNA required to exert a physiological effect is small and that makes it applicable for human therapy. An additional advantage is that nucleic acid can be delivered intranasally to the brain (Hashizume et al., 2008, Neuro-Oncology 10:112-120; Kim et al., 2012, Mol Ther 20(4):829-839). In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects (Kromminga and Schellekens, 2005, Ann NY Acad Sci 1050:257-265).

In certain embodiments, the nucleoside-modified RNA comprises the naturally occurring modified-nucleoside pseudouridine. In certain embodiments, inclusion of pseudouridine makes the mRNA more stable, non-immunogenic, and highly translatable (Karikó et al., 2008, Mol Ther 16:1833-1840; Anderson et al., 2010, Nucleic Acids Res 38:5884-5892; Anderson et al., 2011, Nucleic Acids Research 39:9329-9338; Karikó et al., 2011, Nucleic Acids Research 39:e142; Karikó et al., 2012, Mol Ther 20:948-953; Karikó et al., 2005, Immunity 23:165-175).

It has been demonstrated that the presence of modified nucleosides, including pseudouridines in RNA suppress their immunogenicity (Karikó et al., 2005, Immunity 23:165-175). Further, protein-encoding, in vitro-transcribed RNA containing pseudouridine can be translated more efficiently than RNA containing no or other modified nucleosides (Karikó et al., 2008, Mol Ther 16:1833-1840). Subsequently, it is shown that the presence of pseudouridine improves the stability of RNA (Anderson et al., 2011, Nucleic Acids Research 39:9329-9338) and abates both activation of PKR and inhibition of translation (Anderson et al., 2010, Nucleic Acids Res 38:5884-5892). A preparative HPLC purification procedure has been established that was critical to obtain pseudouridine-containing RNA that has superior translational potential and no immunogenicity (Karikó et al., 2011, Nucleic Acids Research 39:e142). Administering HPLC-purified, pseudourine-containing RNA coding for erythropoietin into mice and macaques resulted in a significant increase of serum EPO levels (Karikó et al., 2012, Mol Ther 20:948-953), thus confirming that pseudouridine-containing mRNA is suitable for in vivo protein therapy.

The present invention encompasses RNA, oligoribonucleotide, and polyribonucleotide molecules comprising pseudouridine or a modified nucleoside. In certain embodiments, the composition comprises an isolated nucleic acid encoding a cell debris-inhibiting or -catabolizing peptide, wherein the nucleic acid comprises a pseudouridine or a modified nucleoside. In certain embodiments, the composition comprises expression vectors, including gene therapy vectors, comprising an isolated nucleic acid encoding a cell debris-inhibiting or -catabolizing peptide, wherein the nucleic acid comprises a pseudouridine or a modified nucleoside.

In one embodiment, the nucleoside-modified RNA of the invention is IVT RNA, as described elsewhere herein. For example, in certain embodiments, the nucleoside-modified RNA is synthesized by T7 phage RNA polymerase. In another embodiment, the nucleoside-modified mRNA is synthesized by SP6 phage RNA polymerase. In another embodiment, the nucleoside-modified RNA is synthesized by T3 phage RNA polymerase.

In one embodiment, the modified nucleoside is m1acp3Ψ (1-methyl-3-(3-amino-3-carboxypropyl) pseudouridine. In another embodiment, the modified nucleoside is m1Ψ (1-methylpseudouridine). In another embodiment, the modified nucleoside is Ψm (2′-O-methylpseudouridine. In another embodiment, the modified nucleoside is m5D (5-methyldihydrouridine). In another embodiment, the modified nucleoside is m3Ψ (3-methylpseudouridine). In another embodiment, the modified nucleoside is a pseudouridine moiety that is not further modified. In another embodiment, the modified nucleoside is a monophosphate, diphosphate, or triphosphate of any of the above pseudouridines. In another embodiment, the modified nucleoside is any other pseudouridine known in the art.

In another embodiment, the nucleoside that is modified in the nucleoside-modified RNA the present invention is uridine (U). In another embodiment, the modified nucleoside is cytidine (C). In another embodiment, the modified nucleoside is adenosine (A). In another embodiment the modified nucleoside is guanosine (G).

In another embodiment, the modified nucleoside of the present invention is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is Ψ (pseudouridine). In another embodiment, the modified nucleoside is Um (2′-O-methyluridine).

In other embodiments, the modified nucleoside is m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²i6A (2-methylthio-N⁶isopentenyladenosine); io⁶A (N⁶-(cis-hydroxyisopentenyl)adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²₂G(N²,N²-dimethylguanosine); m²Gm (N²,2′-O-dimethylguanosine); m²₂Gm (N²,N²,2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵s²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶₂A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶₂Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine).

In another embodiment, a nucleoside-modified RNA of the present invention comprises a combination of 2 or more of the above modifications. In another embodiment, the nucleoside-modified RNA comprises a combination of 3 or more of the above modifications. In another embodiment, the nucleoside-modified RNA comprises a combination of more than 3 of the above modifications.

In another embodiment, between 0.1% and 100% of the residues in the nucleoside-modified of the present invention are modified (e.g. either by the presence of pseudouridine or a modified nucleoside base). In another embodiment, 0.1% of the residues are modified. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 8%. In another embodiment, the fraction is less than 10%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, 0.1% of the residues of a given nucleoside (i.e., uridine, cytidine, guanosine, or adenosine) are modified. In another embodiment, the fraction of the given nucleotide that is modified is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction of the given nucleotide that is modified is less than 8%. In another embodiment, the fraction is less than 10%. In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, a nucleoside-modified RNA of the present invention is translated in the cell more efficiently than an unmodified RNA molecule with the same sequence. In another embodiment, the nucleoside-modified RNA exhibits enhanced ability to be translated by a target cell. In another embodiment, translation is enhanced by a factor of 2-fold relative to its unmodified counterpart. In another embodiment, translation is enhanced by a 3-fold factor. In another embodiment, translation is enhanced by a 5-fold factor. In another embodiment, translation is enhanced by a 7-fold factor. In another embodiment, translation is enhanced by a 10-fold factor. In another embodiment, translation is enhanced by a 15-fold factor. In another embodiment, translation is enhanced by a 20-fold factor. In another embodiment, translation is enhanced by a 50-fold factor. In another embodiment, translation is enhanced by a 100-fold factor. In another embodiment, translation is enhanced by a 200-fold factor. In another embodiment, translation is enhanced by a 500-fold factor. In another embodiment, translation is enhanced by a 1000-fold factor. In another embodiment, translation is enhanced by a 2000-fold factor. In another embodiment, the factor is 10-1000-fold. In another embodiment, the factor is 10-100-fold. In another embodiment, the factor is 10-200-fold. In another embodiment, the factor is 10-300-fold. In another embodiment, the factor is 10-500-fold. In another embodiment, the factor is 20-1000-fold. In another embodiment, the factor is 30-1000-fold. In another embodiment, the factor is 50-1000-fold. In another embodiment, the factor is 100-1000-fold. In another embodiment, the factor is 200-1000-fold. In another embodiment, translation is enhanced by any other significant amount or range of amounts.

In another embodiment, the nucleoside-modified RNA of the present invention is significantly less immunogenic than an unmodified in vitro-synthesized RNA molecule with the same sequence. In another embodiment, the modified RNA molecule is 2-fold less immunogenic than its unmodified counterpart. In another embodiment, immunogenicity is reduced by a 3-fold factor. In another embodiment, immunogenicity is reduced by a 5-fold factor. In another embodiment, immunogenicity is reduced by a 7-fold factor. In another embodiment, immunogenicity is reduced by a 10-fold factor. In another embodiment, immunogenicity is reduced by a 15-fold factor. In another embodiment, immunogenicity is reduced by a 20-fold factor. In another embodiment, immunogenicity is reduced by a 50-fold factor. In another embodiment, immunogenicity is reduced by a 100-fold factor. In another embodiment, immunogenicity is reduced by a 200-fold factor. In another embodiment, immunogenicity is reduced by a 500-fold factor. In another embodiment, immunogenicity is reduced by a 1000-fold factor. In another embodiment, immunogenicity is reduced by a 2000-fold factor. In another embodiment, immunogenicity is reduced by another fold difference.

In another embodiment, “significantly less immunogenic” refers to a detectable decrease in immunogenicity. In another embodiment, the term refers to a fold decrease in immunogenicity (e.g., 1 of the fold decreases enumerated above). In another embodiment, the term refers to a decrease such that an effective amount of the nucleoside-modified RNA can be administered without triggering a detectable immune response. In another embodiment, the term refers to a decrease such that the nucleoside-modified RNA can be repeatedly administered without eliciting an immune response sufficient to detectably reduce expression of the recombinant protein. In another embodiment, the decrease is such that the nucleoside-modified RNA can be repeatedly administered without eliciting an immune response sufficient to eliminate detectable expression of the recombinant protein.

In one embodiment, delivery of nucleoside-modified RNA comprises any suitable delivery method, including exemplary RNA transfection methods described elsewhere herein. In certain embodiments, delivery of a nucleoside-modified RNA to a subject comprises mixing the nucleoside-modified RNA with a transfection reagent prior to the step of contacting. In another embodiment, a method of present invention further comprises administering nucleoside-modified RNA together with the transfection reagent. In another embodiment, the transfection reagent is a cationic lipid reagent.

In another embodiment, the transfection reagent is a lipid-based transfection reagent. In another embodiment, the transfection reagent is a protein-based transfection reagent. In another embodiment, the transfection reagent is a polyethyleneimine based transfection reagent. In another embodiment, the transfection reagent is calcium phosphate. In another embodiment, the transfection reagent is Lipofectin®, Lipofectamine®, or TransIT®. In another embodiment, the transfection reagent is any other transfection reagent known in the art.

In another embodiment, the transfection reagent forms a liposome. Liposomes, in another embodiment, increase intracellular stability, increase uptake efficiency and improve biological activity. In another embodiment, liposomes are hollow spherical vesicles composed of lipids arranged in a similar fashion as those lipids which make up the cell membrane. They have, in another embodiment, an internal aqueous space for entrapping water-soluble compounds and range in size from 0.05 to several microns in diameter. In another embodiment, liposomes can deliver RNA to cells in a biologically active form.

### Polypeptides

In one embodiment, the composition of the invention comprises a cell debris-inhibiting or -catabolizing peptide, as described herein. For example, in one embodiment, the composition comprises at least one of an RNase, a DNase, an ENTPD, and NT5E. In one embodiment, the composition comprises an RNase, a DNase, an ENTPD, and NT5E.

The variants of the polypeptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for detection (for example, His-tag or Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two polypeptides is determined by comparing the amino acid sequence and its conserved amino acid substitutes of one polypeptide to a sequence of a second polypeptide. Variants are defined to include polypeptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence and/or the ability to bind to ubiquitin or to a ubiquitylated protein. The present invention includes amino acid sequences that are at least 60%, 65%, 70%, 72%, 74%, 76%, 78%, 80%, 90%, or 95% similar or identical to the original amino acid sequence. The degree of identity between two polypeptides is determined using computer algorithms and methods that are widely known for the persons skilled in the art. The identity between two amino acid sequences is preferably determined by using the BLASTP algorithm (BLAST Manual, Altschul, S., et al., NCBI NLM NIH Bethesda, Md. 20894, Altschul, S., et al., J. Mol. Biol. 215: 403-410 (1990)).

The polypeptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The polypeptides of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNA_{LYS}), could be modified with an amine specific photoaffinity label.

The term “functionally equivalent” as used herein refers to a polypeptide according to the invention that preferably retains at least one biological function or activity of the specific amino acid sequence of a cell debris-inhibiting or -catabolizing peptide.

A cell debris-inhibiting or -catabolizing peptide, or chimeric protein of the invention may be conjugated with other molecules, such as proteins, to prepare fusion proteins. This may be accomplished, for example, by the synthesis of N-terminal or C-terminal fusion proteins provided that the resulting fusion protein retains the functionality of the cell debris catabolizing or inhibiting peptide.

Cyclic derivatives of the peptides or chimeric proteins of the invention are also part of the present invention. Cyclization may allow the peptide or chimeric protein to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

In a particular embodiment of the invention, the polypeptide of the invention further comprises the amino acid sequence of a tag. The tag includes but is not limited to: polyhistidine tags (His-tags) (for example H6 and H10, etc.) or other tags for use in IMAC systems, for example, Ni²⁺ affinity columns, etc., GST fusions, MBP fusions, streptavidine-tags, the BSP biotinylation target sequence of the bacterial enzyme BIRA and tag epitopes that are directed by antibodies (for example c-myc tags, FLAG-tags, among others). As will be observed by a person skilled in the art, the tag peptide can be used for purification, inspection, selection and/or visualization of the fusion protein of the invention. In a particular embodiment of the invention, the tag is a detection tag and/or a purification tag. It will be appreciated that the tag sequence will not interfere in the function of the protein of the invention.

Accordingly, the polypeptides of the invention can be fused to another polypeptide or tag, such as a leader or secretory sequence or a sequence which is employed for purification or for detection.

The invention also relates to novel chimeric proteins comprising a cell debris catabolizing or inhibiting peptide of the invention fused to, or integrated into, a target protein, and/or a targeting domain capable of directing the chimeric protein to a desired cellular component or cell type or tissue. The chimeric proteins may also contain additional amino acid sequences or domains. The chimeric proteins are recombinant in the sense that the various components are from different sources, and as such are not found together in nature (i.e., are heterologous).

A target protein is a protein that is selected for degradation and for example may be a protein that is mutated or over expressed in a disease or condition. In another embodiment of the invention, a target protein is a protein that is abnormally degraded and for example may be a protein that is mutated or underexpressed in a disease or condition. The targeting domain can be a membrane spanning domain, a membrane binding domain, or a sequence directing the protein to associate with for example vesicles or with the nucleus. The targeting domain can target a cell debris-inhibiting or -catabolizing peptide to a particular cell type or tissue. For example, the targeting domain can be a cell surface ligand or an antibody against cell surface antigens of a target tissue (e.g. neuron or tumor antigens). A targeting domain may target a cell debris catabolizing or inhibiting peptide to a cellular component.

In other embodiments, the composition of the invention comprises a peptidomimetic of a cell debris catabolizing or inhibiting peptide. Peptidomimetics are compounds based on, or derived from, peptides and proteins. The a cell debris inhibiting peptidomimetics of the present invention typically can be obtained by structural modification of a known cell debris catabolizing or inhibiting or catabolizing peptide sequence using unnatural amino acids, conformational restraints, isosteric replacement, and the like. The subject peptidomimetics constitute the continuum of structural space between peptides and non-peptide synthetic structures; a cell debris inhibiting peptidomimetics may be useful, therefore, in delineating pharmacophores and in helping to translate peptides into nonpeptide compounds with the activity of the parent cell debris-inhibiting or -catabolizing peptide.

Antibodies

In one embodiment, the composition of the invention comprises an isolated nucleic acid encoding an antibody, wherein the antibody is an inhibitor of the activity of cell debris. In another embodiment, the composition of the invention comprises an antibody, wherein the antibody is an inhibitor of the activity of cell debris. As will be understood by one skilled in the art, any antibody that can recognize and bind to an antigen of interest is useful in the present invention.

Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, bind to the specific antigens of interest, and they are able to bind the antigen present on Western blots, in solution in enzyme linked immunoassays, in fluorescence activated cells sorting (FACS) assays, in magnetic-activated cell sorting (MACS) assays, and in immunofluorescence microscopy of a cell transiently transfected with a nucleic acid encoding at least a portion of the antigenic protein, for example.

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest. When the antibody used in the invention is humanized, the antibody may be generated as described in Queen, et al. (U.S. Pat. No. 6,180,370), Wright et al., (1992, Critical Rev. Immunol. 12:125-168) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as an epitope on an antigen of interest, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

The invention also includes functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application WO 93/21319 and PCT Application WO 89/09622.

Functional equivalents include polypeptides with amino acid sequences substantially the same as the amino acid sequence of the variable or hypervariable regions of the antibodies. “Substantially the same” amino acid sequence is defined herein as a sequence with at least 70%, preferably at least about 80%, more preferably at least about 90%, even more preferably at least about 95%, and most preferably at least 99% homology to another amino acid sequence (or any integer in between 70 and 99), as determined by the FASTA search method in accordance with Pearson and Lipman, 1988 Proc. Nat'l. Acad. Sci. USA 85: 2444-2448. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′)₂ fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H₂L₂) formed of two dimers associated through at least one disulfide bridge.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Other active agents useful in the treatment of fibrosis include anti-inflammatories, including corticosteroids, and immunosuppressants.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### Treatment Methods

The present invention provides methods of treating stroke in a subject comprising administering an effective amount of a composition comprising an inhibitor of cell debris, described herein. For example, in one embodiment, the method comprises administering to a subject an effective amount of a composition comprising an isolated nucleic acid encoding a catabolizing enzyme. In one embodiment, method comprises administering to a subject an effective amount of a composition comprising a catabolizing enzyme. For example, in one embodiment, the method comprises administering an effective amount of a composition that eliminates at least one of extracellular RNA, extracellular DNA, and extracellular ATP that is released after stroke. The invention includes treatment of cerebral ischemia, subarachnoid hemorrhage, and intracerebral hemorrhage.

In one embodiment, the method comprises administering a composition comprising at least one of an isolated nucleic acid sequence encoding an RNase, an isolated sequence encoding a DNase, an isolated sequence encoding an ENTPD, and an isolated sequence encoding NT5E. In one embodiment, the method comprises administering a composition comprising an isolated nucleic acid sequence encoding an RNase, an isolated sequence encoding a DNase, an isolated sequence encoding an ENTPD, and an isolated sequence encoding NT5E.

In one embodiment, the method comprises administering a composition comprising an isolated nucleic acid encoding at least one of an RNase, a DNase, an ENTPD, and NT5E. In one embodiment, the method comprises administering a composition comprising an isolated nucleic acid encoding an RNase, a DNase, an ENTPD, and NT5E.

In some embodiments, the method comprises administering to the subject a composition comprising nucleoside-modified mRNA encoding at least one of an RNase, a DNase, ENTPD, and NT5E. In one embodiment, the method comprises administering to the subject a composition comprising nucleoside-modified mRNA encoding an RNase, a DNase, ENTPD, and NT5E.

In one embodiment, the method comprises administering a composition comprising at least one of an RNase, a DNase, an ENTPD, and NT5E. In one embodiment, the method comprises administering a composition comprising an RNase, a DNase, an ENTPD, and NT5E.

In another embodiment, the method comprises administering a composition comprising an isolated nucleic acid encoding a peptide that inhibits the activity of cell debris. In one embodiment, the method comprises administering a composition comprising a peptide that inhibits the activity of cell debris.

In certain embodiments, the method of the invention comprises administering to a subject at least one cell debris-inhibiting or -catabolizing peptide described herein (e.g. a catabolizing enzyme described herein) and at least one isolated nucleic acid encoding a cell debris-inhibiting or -catabolizing peptide (e.g. a catabolizing enzyme). This allows for an initial bolus of active peptide as well as a more sustained expression of the peptide as mediated by translation of isolated nucleic acid.

In certain embodiments, the method of the invention allows for sustained presence of the inhibitors of cell debris, described herein, for at least several days post the onset of stroke. However, the method, in certain embodiments, also provides for transient expression, as in certain embodiments, the nucleic acid is not integrated into the subject genome.

In certain embodiments, the method comprises administering nucleoside-modified RNA which provides stable expression of the cell debris inhibitors described herein. In some embodiments, administration of nucleoside-modified RNA results in little to no immune response.

Administration of the compositions of the invention in a method of treatment can be achieved in a number of different ways, using methods known in the art. In one embodiment, the method of the invention comprises systemic administration of the subject, including for example enteral or parenteral administration. In certain embodiments, the method comprises intranasal delivery of the composition. In certain embodiments, intranasal delivery allows for delivery of the composition into the brain. In another embodiment, the method comprises intravenous delivery of the composition. In some embodiments, the method comprises intracerebroventricular delivery of the composition.

It will be appreciated that a cell debris inhibitor of the invention may be administered to a subject either alone, or in conjunction with another therapeutic agent. The inhibitor may also be a hybrid or fusion composition to facilitate, for instance, delivery to target cells or efficacy. In one embodiment, a hybrid composition may comprise a tissue-specific targeting sequence.

The therapeutic and prophylactic methods of the invention thus encompass the use of pharmaceutical compositions comprising a cell debris inhibitor described herein to practice the methods of the invention. The pharmaceutical compositions useful for practicing the invention may be administered to deliver a dose of from ng/kg/day and 100 mg/kg/day. In one embodiment, the invention envisions administration of a dose which results in a concentration of the compound of the present invention from 1 μM and 10 μM in a mammal.

Typically, dosages which may be administered in a method of the invention to a mammal, preferably a human, range in amount from 0.5 μg to about 50 mg per kilogram of body weight of the mammal, while the precise dosage administered will vary depending upon any number of factors, including but not limited to, the type of mammal and type of disease state being treated, the age of the mammal and the route of administration. Preferably, the dosage of the compound will vary from about 1 μg to about 10 mg per kilogram of body weight of the mammal. More preferably, the dosage will vary from about 3 μg to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

The composition may be administered to a mammal within a few minutes, or it may be administered within a few hours, or it may be administered within a few days of the onset of stroke.

In one embodiment, the invention includes a method comprising administering a combination of inhibitors described herein. In certain embodiments, the method has an additive effect, wherein the overall effect of the administering a combination of inhibitors is approximately equal to the sum of the effects of administering each individual inhibitor. In other embodiments, the method has a synergistic effect, wherein the overall effect of administering a combination of inhibitors is greater than the sum of the effects of administering each individual inhibitor.

The method comprises administering a combination of inhibitors in any suitable ratio. For example, in one embodiment, the method comprises administering two individual inhibitors at a 1:1 ratio. In another embodiment, the method comprises administering three individual inhibitors at a 1:1:1 ratio. However, the method is not limited to any number of inhibitors administered at any particular ratio. For example, in one embodiment, the method of the present invention comprises delivering more than one of an RNase, a DNAse, an ENTPD, and NT5E to a subject. In some instances, extracellular RNA, extracellular DNA, and extracellular ATP released after stroke exacerbate the pathological activity of each other. Therefore, in certain embodiments, the present invention comprises catabolizing all of these forms of cell debris simultaneously.

In certain embodiments, the method of the invention comprises delivery of a composition described herein in combination with one or more suitable therapeutic agents. For example, a composition of the invention may be co-administered (administered before, simultaneously, or after) any number of relevant treatment modalities, including but not limited to treatment with agents such as ion channel blockers, glutamate antagonists, glutamate receptor antagonists, enzyme inhibitors, antioxidants, immunosuppressives, and anticoagulants.

The administration of a nucleic acid or peptide inhibitor of the invention to the subject may be accomplished using gene therapy. Gene therapy, which is based on inserting a therapeutic gene into a cell by means of an ex vivo or an in vivo technique. Suitable vectors and methods have been described for genetic therapy in vitro or in vivo, and are known as expert on the matter; see, for example, Giordano, 1996, Nature Medicine 2:534-539; Schaper, 1996, Circ. Res 79:911-919; Anderson, 1992, Science 256:808-813; Isner, 1996, Lancet 348:370-374; Muhlhauser, 1995, Circ. Res 77:1077-1086; Wang, 1996, Nature Medicine 2:714-716; WO94/29469; WO97/00957 and Schaper, 1996, Current Opinion in Biotechnology 7:635-640 and the references cited therein. The polynucleotide codifying the polypeptide of the invention can be designed for direct insertion or by insertion through liposomes or viral vectors (for example, adenoviral or retroviral vectors) in the cell. Suitable gene distribution systems that can be used according to the invention may include liposomes, distribution systems mediated by receptor, naked DNA and viral vectors such as the herpes virus, the retrovirus, the adenovirus and adeno-associated viruses, among others. The distribution of nucleic acids to a specific site in the body for genetic therapy can also be achieved by using a biolistic distribution system, such as that described by Williams (1991, Proc. Natl. Acad. Sci. USA 88:2726-2729). The standard methods for transfecting cells with recombining DNA are well known by an expert on the subject of molecular biology, see, for example, WO94/29469; see also supra. Genetic therapy can be carried out by directly administering the recombining DNA molecule or the vector of the invention to a patient or transfecting the cells with the polynucleotide or the vector of the invention ex vivo and administering the transfected cells to the patient.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### Kinetics of Cell Debris Accumulation and Effects on Pathophysiologic Parameters after Ischemic Brain Damage

The experiments described herein use multiple strategies to determine the kinetics of cell debris accumulation, changes in the capacity of endogenous catabolizing enzymes, and levels of inflammatory and coagulatory mediators and stroke biomarkers in models of ischemic stroke. Analyses are also performed using human stroke patients to corroborate the model system and develop a detailed characterization of ischemic stroke.

For several injury-related diseases, it is recognized that cell debris, molecules that are released from necrotic cells, mediate a series of pathologic alterations, including severe inflammation, bystander cell toxicity and death, endothelial cell dysfunction and edema, and thrombosis leading to further tissue damage that exacerbates outcome (Zeerleder et al., 2003, Crit Care Med 31:1947-1951; Lam et al., 2003, Clin Chem 49:1286-1291; Nakahara et al., 2009, Neurocrit Care 11:362-368; Rykova E Y, Laktionov P P, Vlassov V V, Circulating Nucleic Acids in Health and Disease, in: E. Y. Rykova, Y. Kikuchi, (Eds.), Extracellular Nucleic Acids, Nucleic Acids and Molecular Biology, Springer-Verlag, 2010, pp. 93-128). In the acute phase of ischemic stroke, a large amount of cell death occurs in the infarct core and vasculature. Experiments described herein were designed to perform a comprehensive analysis of the release of stroke-related debris by measuring the amounts of debris in the CSF and circulation, the capacity of the CSF and plasma to degrade debris and the induction of inflammatory mediators, a procoagulant state, and endothelial cell dysfunction. These measurements are performed in a mouse model of middle cerebral artery occlusion (MCAO) and in plasma samples from stroke patients to characterize the kinetics of circulating debris and inflammatory and coagulatory mediators and to verify the model system being employed. These experiments determine how the levels of different cell debris are changing during the disease course of cerebral ischemia and their effect on physiologic pathways. Further, the experiments provide information of how cell debris could be used as a measure for evaluating the effect of delivered catabolizing enzymes.

Measuring Circulating RNA, DNA, ATP, and IL-6

The murine MCOA model of ischemia is described. The middle cerebral artery is blocked for 30 min with 8-0 monofilament silicone-coated nylon surgical suture that is threaded through the external carotid to the internal carotid up to the bifurcation into the MCA and anterior cerebral artery. A suture with a final tip diameter of 0.21-0.22 mm is used for a mouse with body weight of 25-30 g. A laser Doppler probe is placed on the skull surface from time of anesthesia until 15 min after suture removal to monitor blood flow. Rectal temperature is monitored and held constant during surgery using a thermostatically regulated heating pad to prevent artifacts induced by body temperature fluctuations. Permanent blockage is achieved by leaving the filament in place for 24 hr.

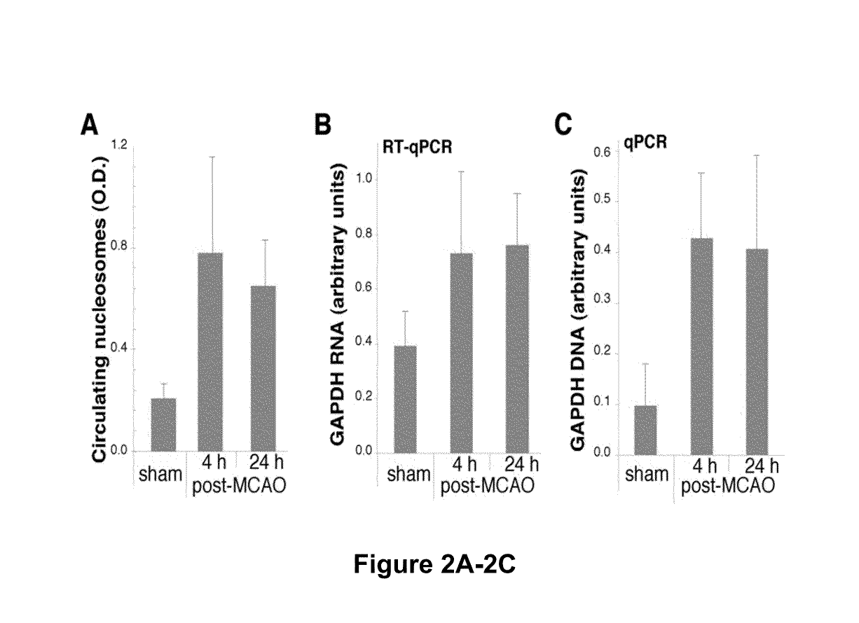

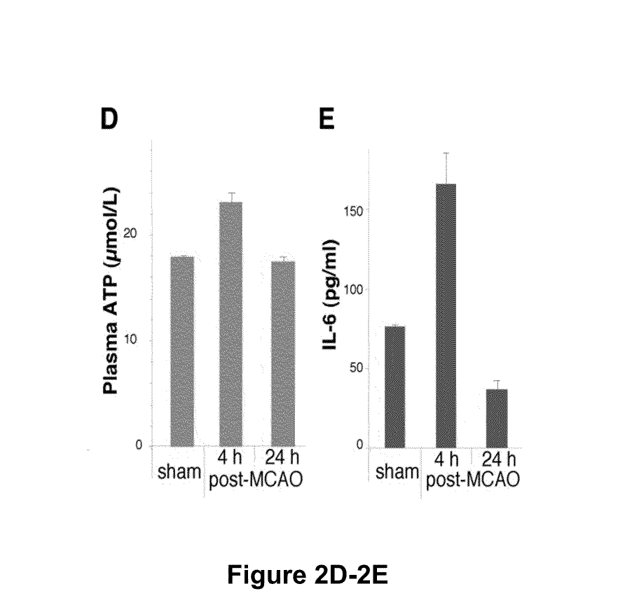

Experiments were performed using 30 minutes of transient MCAO on mice and analyzing their plasma at 4 and 24 h after reperfusion. Samples from sham-operated animals taken 4 h post-surgery were used for comparison. High levels of circulating nucleosomes were measured at both 4 and 24 hours following reperfusion using the Cell Death Detection ELISA that measures histone-complexed DNA fragments by utilizing histone- and DNA-specific antibodies (FIG. 2A). This finding is in agreement with studies reporting elevated nucleosome levels in the plasma of patients with cerebral strokes (Geiger et al., 2006, Cerebrovasc Dis 21:32-37; Geiger et al., 2007, J Neurol 254:617-623; Whiteley et al., 2009, Stroke 40:e380-389). Extracellular nucleic acids were also isolated from plasma and GAPDH-specific RNA and DNA fragments were measured by reverse transcriptase (RT)-real time quantitative (q)PCR and qPCR, respectively. Extracellular RNA and DNA levels were increased at both 4 and 24 h post-MCAO (FIGS. 2B,C). Increased levels of circulating DNA have been found in patients during acute stroke (Rainer et al., 2003, Clin Chem 49:562-569). Until now, no data have been reported on circulating RNA levels in patients or animal models with cerebral ischemia. ATP, measured by a very sensitive bioluminescence assay (ENLITEN, Promega), was detected at significantly higher levels in MCAO animals at 4 h after reperfusion compared to animals subjected to sham operations (FIG. 2D). The amount of increase in ATP, while not large, is in agreement with what has been reported for other diseases (Lader et al., 2000, Clin Physiol 20:348-353). The measurement of circulating ATP is complex and minor alterations in the techniques used can lead to aberrant values (Gorman et al., 2007, Clin Chem 53:318-325). Therefore, an improved approach for obtaining plasma and measuring ATP is also used in these studies (Gorman et al., 2007, Clin Chem 53:318-325). Similarly, IL-6 levels in the plasma were also significantly higher in mice at 4 h post-MCAO compared to animals subjected to sham operations (FIG. 2E). Increased IL-6 levels have been reported for patients with cerebral ischemia, and IL-6 levels correlated with the severity of the disease (Fassbender et al., 1994, J Neurol Sci 122:135-139). Considering that the sham operation also involves surgery, the measured values for sham animals are likely higher than untreated animals. The necessity for an operative procedure to induce an ischemic stroke in the mouse model presents a difference with what is observed in patients.

The total volume of blood required to perform all of the assays in FIG. 2 in duplicate was 20 μl and the addition of other analyses including the measurement of a procoagulant state and additional inflammatory and biomarkers will require 62 μl of plasma. A multiplex Luminex assay is used, which allows that all inflammatory and biomarkers are measured at the same time. The measurement of circulating DNA, RNA, and ATP is performed on as little as 5 μl of blood, which allows sequential measurements at multiple time points after MCAO.

The Extracellular Enzymes Catabolizing ATP, RNA and DNA:

Extracellular ATP is catabolized to AMP by ecto-NTP diphosphohydrolase 1 (ENTPD1), also called ecto-apyrase, and AMP is further catabolize to adenosine by ecto-5′-nucleotidase (NT5E) (Yegutkin, 2008, Biochim Biophys Acta 1783:673-694). Both enzymes are highly expressed on endothelial cells (Yegutkin, 2008, Biochim Biophys Acta 1783:673-694). The RNA- and DNA-catabolizing enzymes RNase1 and DNase1, respectively, are secreted in large quantities by endothelial cells (Landre et al., 2002, J Cell Biochem 86:540-552).

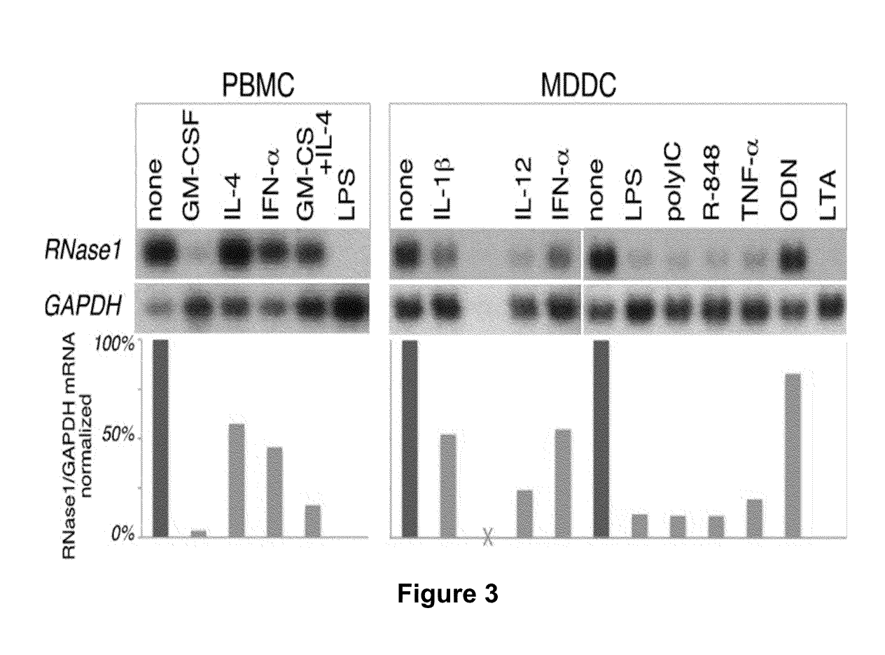

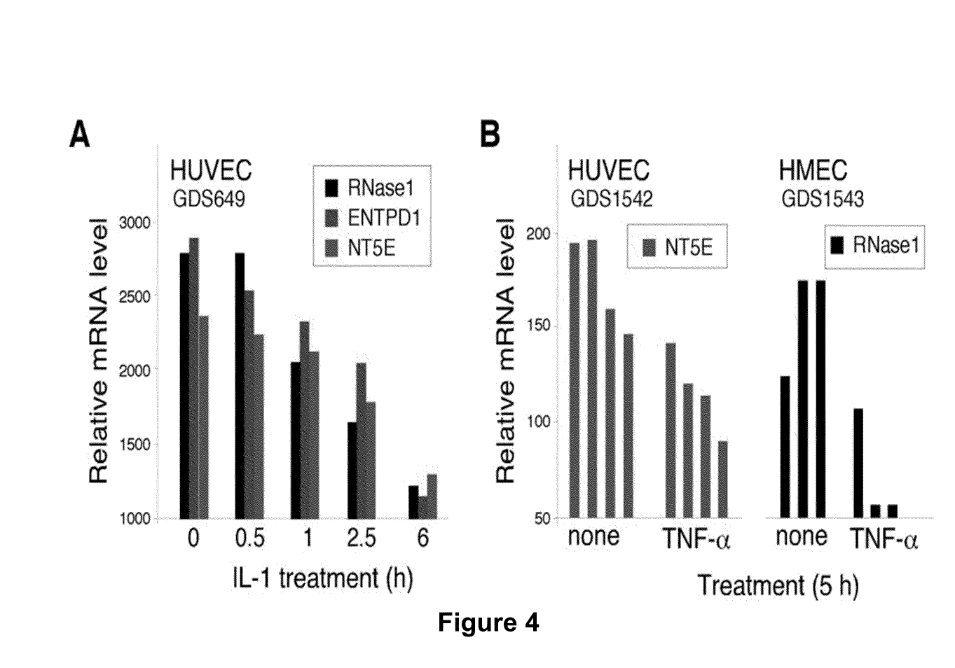

Interestingly, during injury and inflammatory conditions when more debris is circulating, the expression/activity of these catabolizing enzymes are decreased or inhibited. For example, endothelial cell activation leads to a loss of ATP-catabolizing ENTPD1 activity (Robson et al., 1997, J Exp Med 185(1):153-164). Actin and ATP, released by necrotic cells, inhibit activities of DNase1 and RNase1 (Lazarides and Lindberg, 1974, Proc Natl Acad Sci USA 71:4742-4746; Kumar et al., 2003, Biochem Biophys Res Commun 300(1):81-86). In experiments described herein, the expression of RNase1 mRNA by human PBMCs and monocyte-derived dendritic cells (MDDCs) was measured. The results demonstrate that most inflammatory inducers and mediators, including TLR ligands and proinflammatory cytokines, suppressed RNase1 expression (FIG. 3). Further, data mining was performed of microarray analyses performed on endothelial cells (GEO dataset: GDS649, GDS1542, GDS1543). The data shows that IL-1β treatment inhibited expression of RNase1, ENTPD1 and NT5E in human umbilical vein endothelial cells (HUVECs), while TNF-α inhibited NT5E and RNase1 expression in HUVECs and human microvascular endothelial cells (HMECs), respectively (FIG. 4).

The inflammatory and pathologic activities of extracellular ATP, RNA and DNA, and inflammation-induced alterations in the levels of their catabolizing enzymes, provide a rational to measure their levels in the blood during stroke. Thus far, only extracellular levels of DNA have been measured in plasma of ischemic stroke patients and elevated values correlated with a poor prognosis (Geiger et al., 2006, Cerebrovasc Dis 21:32-37; Geiger et al., 2007, J Neurol 254:617-623; Rainer et al., 2003, Clin Chem 49:562-569; Whiteley et al., 2009, Stroke 40:e380-389). Extracellular RNA, which has, until now, not been measured in cerebral ischemia, is also elevated as demonstrated herein, and also is likely to play a damaging role. This is supported by observations that high expression levels of the RNA sensors TLR7 and TLR8 in blood cells of patients with cerebral ischemia associated with poor outcome, increased infarct volume and higher levels of inflammatory IL-1β and IL-6 secretion (Brea et al., 2011, Clin Immunol 139:193-198) and treatment with RNase1 protein in a stroke model had efficacy (Walberer et al., 2009, Curr Neurovasc Res 6:12-19).

Measuring Extracellular ATP, RNA, DNA, RNase, DNase, Inflammatory Mediators, and Stroke Biomarkers after Brain Ischemia

Murine Experiments.

Groups of female C57/BL6 mice 10- or 40-week-old are subjected to 30 minutes of transient MCAO (Table 1) or 10-week-old mice will be exposed to permanent MCAO, as previously performed in rats (Muramatsu et al., 2006, Brain Res 1097:31-38; Katsumata et al., 1999, Eur J Pharmacol 372:167-174; Katsumata et al., 2003, Brain Res 969:168-174). Blood samples taken prior to the MCAO (7 days) and at 4 h, 1, 2, 3 and 4 days after MCAO are analyzed for debris and their catabolizing enzymes, as listed in Tables 2 and 3. Blood for the measurement of a procoagulant state and inflammatory mediators, as well as stroke biomarkers is obtained 24 h after MCAO and at the end of the experiment, day 4. For the measurement of debris and catabolizing enzymes at each time point, 10 μl of blood is collected from the tail vein using microcapillary tubes and the plasma is obtained following centrifugation, as previously described (Karikó et al., 2012, Mol Ther 20:948-953). For the measurement of Factor Xa and Thrombin activity and inflammatory mediators and stroke biomarkers, 140 μl of blood is obtained. These amounts of blood allow for all analyses in duplicate on each animal (Table 2). Cerebrospinal fluid (CSF) is obtained from mice in groups of 5, before and 4 h, 1, 2, 3 and 4 days after MCAO. For this experiment, 6 groups of mice is used and CSF is taken only once from each mouse. CSF is collected as demonstrated previously (Liu and Duff, 2008, J Vis Exp (21) pii:960), but the capillary tube is mounted to improve precision of its handling, thus avoiding blood contamination of the CSF (Liu et al., 2012, Am J Physiol Regul Integr Comp Physiol 303(9):R903-8). CSF is analyzed as described for plasma.

TABLE 1
Experimental protocols

| | | Animals/ | Injury | Analyzed | | |
|---|---|---|---|---|---|---|
| Exp. | Groups | group | model | sample | Time point | Measured parameters |
| 1 | 1 | 10 | MCAO | plasma | D-7, 4h, D1, D2, | ATP, RNA, DNA, RNase, |
| | 1 (old mice) | 10 | MCAO | plasma | D3, D4 | DNase, cytokines, |
| | 1 | 10 | Permanent- | plasma | | Luminex and |
| | | | MCAO | | | procoagulants (D1 & D4) |
| | 6 | 5 | MCAO | CSF | | |

ATP is measured by bioluminescence detection with modifications to improve sensitivity and exclude contamination, ATP degradation or uptake by cells or platelets, as described (Gorman et al., 2007, Clin Chem 53:318-325). RNA and DNA levels are measured as described elsewhere herein. To determine RNase and DNase activity in the plasma and CSF, dual-labeled (6-FAM (fluorescent) and BHQ-1 (quencher)) RNA (RNaseAlert, Ambion) and custom made DNA probes, are used respectively. A FRET assay is performed, similar to that performed for the measurement of RNase L (Anderson et al., 2011, Nucleic Acids Research 39:9329-9338). The measured cytokines include ones activated by cell debris. Biomarkers associated with stroke (Wunderlich et al., 2004, J Neurol Sci 227:49-53; Whiteley et al., 2012, Stroke 43:86-91; Whiteley et al., 2011, Cerebrovasc Dis 32:141-147; Giannakoulas et al., 2005, Angiology 56:723-730; Foerch et al., 2005, Arch Neurol 62:1130-1134; Altamura et al., 2009, Stroke 40:1282-1288; Datta et al., 2011, J Proteome Res 10(11):5199-5213) are also measured as additional measures to follow damage and effect of catabolizing enzyme treatment. The following proinflammatory cytokines and biomarkers are measured by high throughput Luminex multiplex assays: IL-1β, IL-6, IL-8, TNF-α, IFN-α, IFN-β, HMGB1, transferrin (Datta et al., 2011, J Proteome Res 10(11):5199-5213; Altamura et al., 2009, Stroke 40:1282-1288), N-terminal pro-brain natriuretic peptide (Whiteley et al., 2011, Cerebrovasc Dis 32:141-147; Giannakoulas et al., 2005, Angiology 56:723-730), 5-100B (Foerch et al., 2005, Arch Neurol 62:1130-1134; Wunderlich et al., 2004, J Neurol Sci 227:49-53), and neuron-specific enolase (Wunderlich et al., 2004, J Neurol Sci 227:49-53). Measures of a procoagulant state, activated Factor Xa and Thrombin, are performed using chromogenic and fluorogenic assays, respectively (Shibamiya A et al., 2009, Blood 113:714-722; van Berkel et al., 2012, ChemMedChem 7:606-617). Animals are sacrificed on day 4 and the infarct is identified by histological evaluation of serial hematoxylin- and eosin-stained coronal sections of the brain (Brown and Brierley, 1972, J Neurol Sci 16(1):59-84). Infarct volume is calculated based on the area of damage at each section and distance between sections (Frieler et al., 2011, Stroke 42:179-185).

Groups of 5 or 10 animals are used. With 5 animals per group, assuming a standard deviation of 30% for analyses, a 2.0 standard deviation difference is able to be to detected between groups. With 10 animals per group, which is used in experiments to demonstrate efficacy, a 1.3 standard deviation difference is able to be detected between groups. These differences are clinically significant.

TABLE 2
Plasma requirements for analyses

| | assay | plasma | | Days post MCAO | | | | |
|---|---|---|---|---|---|---|---|---|
| | dilution | volume (μl) | −7 | 0.17 | 1 | 2 | 3 | 4 |
| ATP | 1:100 | 0.01 | X | X | X | X | X | X |
| nucleosome | 1:20 | 1 | X | X | X | X | X | X |
| RNA | 1:100 | 0.5 | X | X | X | X | X | X |
| DNA | 1:100 | 0.5 | X | X | X | X | X | X |
| Luminex | 1:1* | 50 | X | | X | | | X |
| Coagulation | 1:3 | 10 | X | | X | | | X |
| Total plasma needed (μl) | | | 62 | 2 | 62 | 2 | 2 | Terminal |

*higher dilutions used for certain proteins

Human Studies.

To verify the quality of the murine model system described above, and to obtain detailed data on ischemic stroke in humans that fully characterize these parameters to help to begin to understand their pathophysiological role, measurements of debris, inflammatory cytokines, stroke biomarkers, procoagulant activity and catabolizing enzymes are performed in patients admitted with MCA strokes. Further, patients treated with tPA are also analyzed, which better approximate the murine model, i.e., ischemic stroke followed by reperfusion.

Subjects are identified and subjected to the criteria in Table 4. CT or MRI scans are obtained at identification/admission. Stroke size is determined using Alberta Stroke Program Early CT Score (ASPECTS) (Pexman et al., 2001, AJNR Am J Neuroradiol. 22(8):1534-1542). Controls are obtained from outpatient clinics.

A cross-sectional case control study is performed of patients with documented MCA occlusion compared to controls adjusted for age, sex, and disease modifiers (e.g., diabetes, coronary artery disease, hypertension, hypercholesterolemia, tobacco and alcohol use, and obesity). Blood samples are collected from 50 MCA stroke patients, 15 MCA stroke patients treated with tPA, and 25 matched controls (Table 3). Inclusion and exclusion criteria is similar to those used in the ATLANTIS study (Clark et al., 1999, JAMA 282:2019-2026) (Table 4). Five milliliters of anticoagulated blood is obtained as soon as possible but less than 8 h after appearance of symptoms and then daily until discharge. Blood from tPA-treated patients is obtained prior to treatment and then daily until discharge. Analyses, as described for mouse plasma, are performed.

Studies of stroke patients demonstrate that MCA strokes reperfuse in approximately 50% by 7 days but the clinical significance of this is unclear. A very small subset of stroke patients spontaneously reperfuse very early after the event and demonstrate clinical improvement (Bowler et al., 1998, J Neurol Neurosurg Psychiatry 64(1):90-97) and are easily identified clinically. These subjects are grouped with the tPA treated group for analyses.

Analysis begins by comparing all stroke patients, excluding tPA-treated, to controls for each measured parameter using one-way analysis of variance (ANOVA) with Bonferroni correction. Each parameter measured was further analyzed based on the size of the stroke, divided into ASPECTS scores: 7-9, 4-6, and 0-3, using linear regression and Spearman correlation. tPA treated subjects are evaluated by: 1) including them in the linear regression analyses as the smallest sized stroke group and 2) comparing changes in pre-tPA to post-tPA measurements compared to values of untreated stroke patients. Subgroups of human stroke patients are then compared to the different groups of murine MCAO using one-way ANOVA. Assuming an equal distribution of stroke sizes, a difference of less than 2 standard deviations for each of the analyses is able to be detected using Bonferroni correction in the human trial.

The analyses of samples from humans with ischemic stroke is a descriptive study, but it serves to deepen the understanding of the kinetics of release of cell debris and their effect on inflammatory mediators, biomarkers and procoagulants. It also provides data for the design of future studies and determines whether other types of debris should also be targeted for intervention.

The data from patients is analyzed based on the size of their stroke. Patients treated with tPA are also compared to controls and untreated stroke patients. This data is useful in ensuring that the murine model used a good model. Multiple aspects of the model can be altered to more closely mimic the levels and kinetics of cell debris, catabolizing enzymes, and inflammatory mediators, including the duration of ischemia. When samples from 70 ischemic stroke patients were analyzed, extracellular DNA was found to be 5-times higher in the plasma from those who died compared to those who survived (Rainer et al., 2003, Clin Chem 49:562-569). Plasma DNA content had 100% sensitivity and 74% specificity to predict early mortality. Nucleosome content in 63 cerebral stroke patients also strongly correlated with stroke severity (Geiger et al., 2006, Cerebrovasc Dis 21:32-37). Measured plasma components of mice and patients which are significantly elevated and potentially playing a pathologic role, dictate the design of mRNAs coding for catabolizing enzymes or dominant negative truncated products to lower the impact of those types of debris.

TABLE 3
Human stroke patient study

| | | Measured parameters | | | | | |
|---|---|---|---|---|---|---|---|
| | | | | | | | Cytokines, |
| | | | | | | | procoagulants, |
| Patient # | Disease | ATP | RNA | DNA | RNase | DNase | biomarkers |
| 50 | Ischemic Stroke | + | + | + | + | + | + |
| 15 | Ischemic Stroke + tPA | + | + | + | + | + | + |
| 25 | Control | + | + | + | + | + | + |

TABLE 4
Inclusion and exclusion criteria of subjects
Inclusion Criteria

| | |
|---|---|
| a. | Age 18 through 79 years. |
| b. | Clinical diagnosis of middle cerebral artery stroke causing a |
| | measurable neurological deficit. Ischemic stroke is defined as |
| | an event characterized by the sudden onset of an acute focal |
| | neurological deficit presumed to be due to cerebral ischemia |
| | after exclusion of hemorrhage by CT or MRI scan. |

Exclusion Criteria

| | |
|---|---|
| a. | Coma, severe obtundation, fixed eye deviation, or complete |
| | hemiplegia. |
| b. | Patient has only minor stroke symptoms (i.e., 4 points on the |
| | National Institutes of Health Stroke Scale and normal speech |
| | and visual fields) or major symptoms that are rapidly improving. |
| c. | Presumed septic embolus. |
| d. | Pregnancy, lactation, or parturition within the previous 30 days. |
| e. | Onset of symptoms greater that 8 hrs prior to admission |
| f. | Diagnosis of active malignancy. |
| g. | High-density lesion on CT or MRI consistent with hemorrhage |
| | of any degree or subarachnoid hemorrhage. |
| h. | Immunomodulatory therapy |

### Example 2

### Application of mRNAs Encoding Cell-Debris-Catabolizing Enzymes to Limit Damage and Improve Functional Recovery after Cerebral Ischemia

The delivery of enzymes to catabolize one type of cellular debris has been shown to reduce inflammation in an ischemia-reperfusion injury model. However, no therapy targeting most or all of the principal types of cell debris with effective delivery systems has been performed for any injury or inflammatory models, including brain ischemia.

Experiments described here examine the effects of the delivery of in vitro transcribed-mRNAs encoding enzymes that catabolize extracellular ATP, RNA and DNA to mice subjected to transient middle cerebral artery occlusion. The mRNAs serve as templates for continuous enzyme production in vivo lasting several days. mRNAs are delivered by different routes and schedules. To determine the effectiveness of these treatments, time course analyses are performed to measure debris levels; debris-catabolizing activity and inflammatory, coagulatory and biomarker levels in plasma. Infarct volume and functional recovery are determined and correlated with outcome measures and treatment regiments.

The targeting of injury-related cell debris in the extracellular space for intervention as a therapeutic approach has been considered for many diseases, especially when severe inflammation plays a pathophysiologic role. The use of DNase1 as a therapeutic approach has the longest history. High doses of DNase were injected intrathecally to liquefy DNA in patients with pneumococcal meningitis (Johnson et al., 1959, N Engl J Med 260(18):893-900), intravenously into patients with gout (Ayvazian and Ayvazian, 1960, N Engl J Med 263:999-1002) and parenterally to treat pulmonary abscesses (Ayvazian et al., 1957, Am Rev Tuberc 76:1-21). DNase was also used to treat patients with SLE (Davis et al., 1999, Lupus 8:68-76). At present, the only FDA approved clinical use of DNase is to reduce sputum viscosity in cystic fibrosis patients (Ulmer et al., 1996, Proc Natl Acad Sci USA 93:8225-8229). RNase1 and soluble forms of ENTPD1 and NT5E have been used successfully in animal models to degrade their corresponding substrates in the extracellular space (Thompson et al., 2004, J Exp Med 200:1395-1405; Walberer et al., 2009, Curr Neurovasc Res 6:12-19; Sugimoto et al., 2009, J Thorac Cardiovasc Surg 138:752-759; Drosopoulos et al., 2010, Thromb Haemost 103:426-434; Straub et al., 2011, Arterioscler Thromb Vasc Biol 31:1607-1616; Reutershan et al., 2009, FASEB J 23:473-482; Chen et al., 2014, J Am Heart Assoc 3: e000683), but have not entered clinical trials.

The previous studies that targeted a single type of cell debris demonstrated significant but incomplete protection (Walberer et al., 2009, Curr Neurovasc Res 6:12-19; Sugimoto et al., 2009, J Thorac Cardiovasc Surg 138:752-759; Straub et al., 2011, Arterioscler Thromb Vasc Biol 31:1607-1616; Reutershan et al., 2009, FASEB J 23:473-482). It is believed that the type of injury, which determines the source of the cell debris; the location of the injury, which determines the effect of the various pathways activated; and the local and systemic host response to the debris are important in selecting what should be catabolized for maximal effect. Targeting more than one type of cell debris for elimination is critical, considering the pleiotropic effects of those molecules. Extracellular RNA, DNA and ATP induce inflammation through a wide-range of danger-sensing receptors, and they also potentiate each other's toxicities, e.g., ATP inhibits RNase and DNase activity (Kumar et al., 2003, Biochem Biophys Res Commun 300(1):81-86; Lazarides and Lindberg, 1974, Proc Natl Acad Sci USA 71:4742-4746) thus simultaneous elimination of debris will have an added or synergistic benefit. Therefore, for ischemic strokes, it was selected to eliminate extracellular ATP, RNA and DNA. The degradation of these debris specifically reduce vascular leakage and edema (Thompson et al., 2004, J Exp Med 200:1395-1405; Fischer et al., 2007, Blood 110:2457-2465; Fischer et al., 2009, FASEB J 23(7):2100-2109), thrombosis (Nakazawa et al., 2005, Biochem J 385:831-838; Kannemeier et al., 2007, Proc Natl Acad Sci USA 104:6388-6393; Straub et al., 2011, Arterioscler Thromb Vasc Biol 31:1607-1616; Shibamiya et al., 2009, Blood 113:714-722), and the triggering of additional bystander necrosis mediated by the activation of TLR3 and TLR9 (Shibamiya et al., 2009, Blood 113:714-722; Vanlangenakker et al., 2012, Cell Death Differ 19(1):75-86; Zhang et al., 2010, Nature 464:104-107; Chen et al., 2014, J Am Heart Assoc 3: e000683).

In conditions where highly elevated levels of extracellular HMGB1 are measured and it is determined that it plays a role in the pathophysiology of stroke-induced damage, HMGB1 may be targeted by delivering mRNA encoding BoxA fragment, which neutralizes HMGB1 (Muhammad et al., 2008, J Neurosci 28:12023-12031).

In conditions where peroxiredoxins are found to be elevated and it is determined that they play a pathologic role, monoclonal antibody-encoding mRNAs targeting peroxiredoxins (Prx5 and Prx6 (Shichita et al., 2012, Nat Med 18:911-917)) may be delivered. Single mRNA constructs have been created encoding both heavy and light chains of antibody separated by a P2A sequence (Kim et al., 2011, Plos One 6:e18556) that allows equimolar generation of two unique proteins.

As described herein, mRNAs encoding murine ENTPD1, NT5E, RNase1 and DNase1 are used to treat mice subjected to MCAO. Using these mRNAs, degradation of extracellular ATP to adenosine, and RNA and DNA to their composing nucleotides is induced. Naturally, ENTPD1 and NT5E are ectoenzymes expressed on the cell surface, primarily on endothelial cells in the vasculature (Colgan et al., 2006, Purinergic Signal 2(2):351-360; Knowles, 2011, Purinergic Signal 7(1):21-45; Marcus et al., 2003, J Pharmacol Exp Ther 305:9-16). Following guides from the literature (Straub et al., 2011, Arterioscler Thromb Vasc Biol 31:1607-1616; Drosopoulos et al., 2010, Thromb Haemost 103:426-434), the coding sequences of mouse ENTPD1 and NT5E are re-engineered by inserting sequences corresponding to the mouse IL-2 secretion signal to their 5′-end and eliminating sequences coding for the short transmembrane domain or the region that binds to GPI, respectively. In some experiments, the mRNAs are delivered intravenously, since most of the damage-related debris is expected to be present in the circulation, exudating from dying endothelial cells during reperfusion following ischemia. Under normal conditions, small amounts of released RNA and DNA degrade quickly in the extracellular space, but in the setting of injury, the levels of RNase and DNase are reduced and their activities are inhibited (FIGS. 3 and 4) (Kumar et al., 2003, Biochem Biophys Res Commun 300(1):81-86; Lazarides and Lindberg, 1974, Proc Natl Acad Sci USA 71:4742-4746).

Since RNA has been shown to enter the brain through the intranasal route (Kim et al., 2012, Mol Ther 20(4):829-839; Lorenzi et al., 2010, BMC Biotechnol 10:77; Kanazawa et al., 2013, Biomaterials 34, 9220-9226) the enzyme-encoding mRNAs are also delivered by this route. This aids in the degradation of the high amount of ATP, which has been detected in the penumbra, and likely released from the surrounding cells dying in the infarct core (Hattori et al., 2010, Antioxid Redox Signal 13:1157-1167), as well as high local concentrations of DNA and RNA. The impact of the mRNA treatment on mice subjected to MCAO is determined by measuring levels of debris, proinflammatory cytokines, stroke biomarkers, and a procoagulant state in the plasma, testing neurobehavioral deficits, determining the infarct size and measuring cerebral edema (Xu et al., 2011, J Biomed Opt 16(6):066020). Experiments are performed using a photoacoustic tomography, or alternatively, a small animal MRI device.

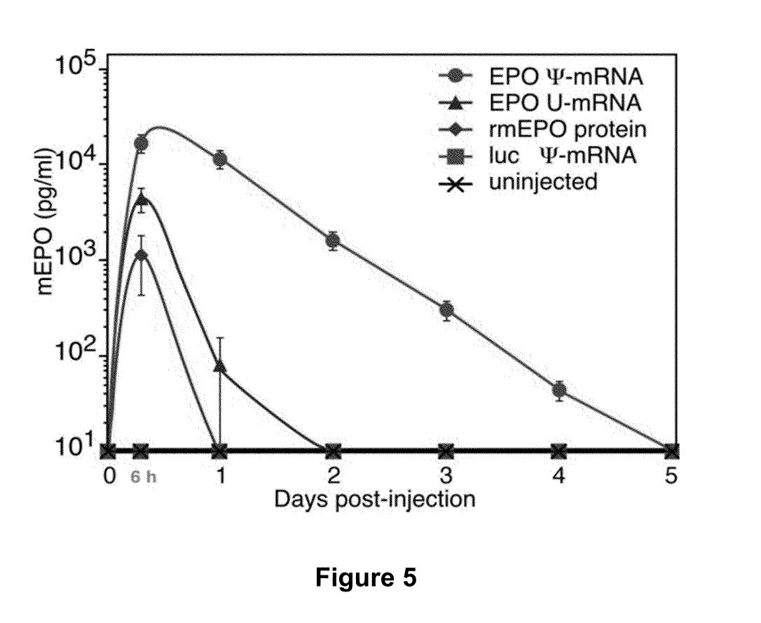

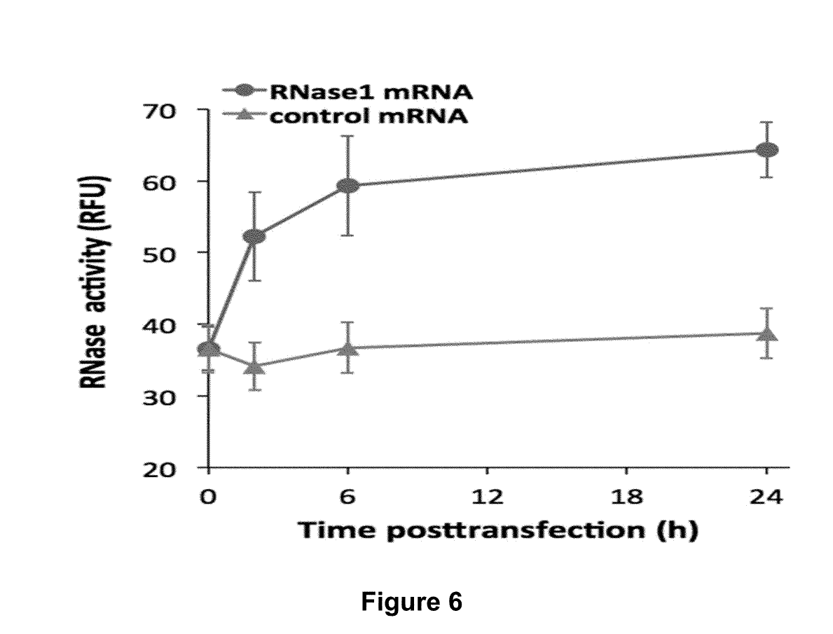

Dose finding for the amount of mRNA encoding catabolizing enzymes is performed in two manners. The calculated amounts of debris found in the circulation after MCAO is used to set up a model that allow for the efficient determination of the amounts of each mRNA needed to catabolize the debris based on the peak level and duration of debris post-MCAO. An efficient method to overexpress functional proteins in vivo by delivering their encoding nucleoside modified mRNA has been developed (Karikó et al., 2008, Mol Ther 16:1833-1840; Karikó et al., 2012, Mol Ther 20:948-953). High levels of protein are produced for an extended amount of time (FIG. 5) (Karikó et al., 2012, Mol Ther 20:948-953) with no activation of inflammatory pathways or genetic alterations as observed in viral gene therapy (Raper et al., 2003, Mol Genet Metab 80:148-158; Morgan et al., 2010, Mol Ther 18:843-851). Experiments were performed which demonstrated that delivery of mRNA encoding RNase1 significantly increases RNase activity in the plasma for at least 24 hrs, when HPLC-purified, pseudouridine-containing mRNA encoding mouse RNase1 was delivered to mice (FIG. 6).

In one experiment, TransIT-complexed mRNAs coding for ENTPD1, NT5E, RNase1 and DNase1 is injected by the i.v. route. A mixture of ATP, RNA and DNA is then injected i.v. to mimic released debris as determined from earlier studies. While it would seem contradictory to deliver mRNA as a therapeutic to treat an excess of extracellular RNA, the presence of the modified nucleoside pseudouridine in the purified mRNA allows it to avoid activating innate immune sensors and complement (Karikó et al., 2008, Mol Ther 16:1833-1840; Anderson et al., 2010, Nucleic Acids Res 38:5884-5892; Anderson et al., 2011, Nucleic Acids Research 39:9329-9338; Karikó et al., 2011, Nucleic Acids Research 39:e142; Karikó et al., 2012, Mol Ther 20:948-953) and its complexing to TransIT protects the mRNA from binding coagulation factors and proteins involved in endothelial dysfunction. The experiment is performed such that doses of the injected mRNAs are adjusted based on prior results to determine the optimal range of amounts of mRNA. Clearance of the injected mimic molecules is followed by measuring the parameters described elsewhere herein, at 4 h, and at 1, 2, 3 and 4 days post-injection (Tables 2, 3 and 6). The goal is to deliver a sufficient amount of mRNA to eliminate the injury mimic molecules at the earliest time. These experiments provide approximate amounts of catabolizing enzyme-encoding mRNA needed to clear exogenously delivered debris. Circulating levels of immune activation and a procoagulant state are monitored as an indicator that the debris is being effectively degraded. Through the measurement of residual debris and inflammatory and coagulatory markers, amount of each debris-catabolizing mRNA needed is modulated. While this approach replicates the peak level of debris, but not the duration of debris release, final dose finding is performed in mice subjected to MCAO. Additional types of debris that is found to be important and continue to mediate inflammation, coagulation, and vascular dysfunction are also targeted for degradation. Such additional factors are made apparent as a complete and rapid loss of targeted debris (ATP, RNA and DNA) after treating the MCAO mice, but continued measured elevation of inflammatory markers, stroke biomarkers, a procoagulant state, and cerebral edema. In these instances, additional mRNAs encoding neutralizers or catabolizing enzymes are delivered, such as BoxA fragment that neutralizes HMGB1 (Muhammad et al., 2008, J Neurosci 28:12023-12031), and peroxiredoxin- (Shichita et al., 2012, Nat Med 18:911-917) and histone-neutralizing antibodies (Huang et al., 2011, Hepatology 54:999-1008).

Mice are pre-treated with mRNAs encoding catabolizing enzymes or control mRNA followed by MCAO. The studies continue dose finding using circulating levels of debris, inflammatory and biomarkers, procoagulant, animal neurobehavioral deficit, and infarct size along with the measurement of debris and inflammatory markers in the CSF. CSF is collected, as described elsewhere herein, at a single time point post MCAO, and analyzed (Table 1). The amounts of mRNA encoding catabolizing enzymes are optimized as efficacy studies are performed. For example, in conditions where it is observed that circulating type 1 interferons remain elevated and RNA debris in the CSF or plasma are also increased, the amount of RNase1 encoding mRNA is raised. Analyses have the ability to determine if an excess of catabolizing enzymes are delivered, which could result loss of benefit due to toxicity or the loss of beneficial effects mediated by debris release. The ultimate end-points of the analyses are animal functional outcome and infarct size.

The accumulation of debris occurs in 2 distinct sites leading to different pathophysiologic processes. The process of ischemia-reperfusion leads to diffuse endothelial cell death and systemic debris accumulation leading to the activation of inflammation, increased coagulation and endothelial cell dysfunction. At the site of the infarct, high levels of debris are released locally leading to very high levels of surrounding inflammation with edema and expansion of local damage. Released RNA also lead to increased endothelial cell dysfunction further resulting in extracellular fluid accumulation. The delivery of mRNA by the intravenous route primarily leads to systemic protein delivery with an unknown amount of the catabolic enzymes reaching the brain and infarct, which are measured in CSF samples. mRNA can also be directly delivered to the CSF, which would not be an ideal approach for a therapeutic, or it can be delivered intranasally that results in protein production in the brain (Lorenzi et al., 2010, BMC Biotechnol 10:77). It is believed that systemic delivery will target many of the most dangerous and damaging effects of stroke-released debris, increased coagulation and complement activation, inflammation and vascular dysfunction but delivery to the brain may have additional beneficial effects. The goal of these experiments is to test which delivery route(s) are the most effective to eliminate debris and lower inflammation, coagulation, cerebral edema and infarct volume and improve functional outcome. The mRNAs are administered to mice prior to MCAO by 3 routes or combinations of routes, i.v., intranasal (i.n.) and intracerebroventricularly (i.c.v.). Mice are followed for coagulatory, inflammatory and biomarkers, debris, infarct volume, and neurobehavioral deficit.

A therapeutic agent or agents needs to be delivered after an ischemic stroke and the duration of time after the event it remains efficacious is an important determinant. tPA, the main current therapeutic for ischemic stroke, needs to be delivered within 3 h of the onset of symptoms. These experiments determine the effect of delayed mRNA treatment (0, 1, 2, 4, or 8 h post MCAO) on inflammatory, coagulatory and biomarkers, debris, infarct volume, and neurobehavioral deficit with the goal of identifying the amount of time after the onset of an ischemic stroke the delivery of debris-catabolizing enzymes remains effective. Delayed treatments are evaluated for all of the routes described elsewhere herein. The proteins encoded by the mRNAs are all murine in origin and their elevated expression has not been associated with detrimental effects, which allows for the delivery of increased amount of mRNA to test if this will prolong the post stroke time when applying the mRNA treatment remains effective.

Cylinder testing is performed to test neurobehavioral deficits caused by MCAO, as previously described (Sansing et al., 2011, Ann Neurol 70:646-656). The modified Bederson score (Bederson et al., 1986, Stroke 17:472-476) is used to determine global neurological function. Motor function and coordination are evaluated by the grip test (Moran et al., 1995, Proc Natl Acad Sci USA 92(12):5341-5345).

Analyses presented herein compare the effects of different treatments (amount, route, time, potentially additional mRNAs) on a variety of measures of outcome. One-way ANOVA are used for normally distributed data and the Wilcoxon-Mann Whitney or Kruskal Wallis test are used for non-normal distributed data. For differences in multiple comparisons Bonferroni correction is used.

In conditions where other types of debris (HMGB1, histones, peroxiredoxins) need to be catabolized or neutralized, proteins have been identified with these functions and can be included (BoxA and neutralizing mAbs) in therapies. It was observed that in vivo protein production from mRNA begins within minutes following its delivery, but in certain conditions, an initial bolus of functional protein is needed. The combination of protein plus encoding mRNA can be used to achieve high initial concentrations of functional protein followed by continuous production over days. The enzymes that are being delivered are present in the extracellular space. Thus, unlike tPA, their delivery is not expected to have any deleterious effects.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A composition for treating stroke comprising at least one isolated nucleic acid encoding at least one cell debris inhibitor.

2. The composition of claim 1, wherein the at least one cell debris inhibitor is a catabolizing enzyme.

3. The composition of claim 1, wherein the at least one cell debris inhibitor is at least one selected from the group consisting of an RNase, a DNase, an ENTPD, and NT5E.

4. The composition of claim 1, wherein the at least one isolated nucleic acid comprises in vitro transcribed RNA.

5. The composition of claim 1, wherein the at least one isolated nucleic acid comprises nucleoside-modified RNA.

6. The composition of claim 1, wherein the at least one isolated nucleic acid comprises pseudouridine.

7. The composition of claim 1, wherein the composition comprises an isolated nucleic acid encoding an RNase, an isolated nucleic acid encoding a DNase, an isolated nucleic acid encoding an ENTPD, and an isolated nucleic acid encoding NT5E.

8. The composition of claim 1, wherein the composition further comprises an isolated nucleic acid encoding an inhibitor of HMGB1.

9. The composition of claim 1, wherein the composition further comprises an isolated nucleic acid encoding an inhibitor of peroxiredoxins.

10. A composition of claim 1, wherein the composition further comprises a cell debris-inhibiting or -catabolizing peptide.

11. A composition for treating stroke comprising at least one cell debris-inhibiting or -catabolizing peptide.

12. The composition of claim 11, wherein the at least one cell debris-inhibiting or -catabolizing peptide is a catabolizing enzyme.

13. The composition of claim 11, wherein the at least one cell debris-inhibiting or -catabolizing peptide is at least one selected from the group consisting of an RNase, a DNase, an ENTPD, and NT5E.

14. The composition of claim 11, wherein the composition comprises an RNase, a DNase, an ENTPD, and NT5E.

15. The composition of claim 11, wherein the composition further comprises an inhibitor of HMGB1.

16. The composition of claim 11, wherein the composition further comprises an inhibitor of peroxiredoxins.

17. A method of treating stroke comprising administering an effective amount of a composition comprising at least one isolated nucleic acid encoding at least one cell debris inhibitor.

18. The method of claim 17, wherein the at least one cell debris inhibitor is a catabolizing enzyme.

19. The method of claim 17, wherein the at least one cell debris inhibitor is at least one selected from the group consisting of an RNase, a DNase, an ENTPD, and NT5E.

20. The method of claim 17, wherein the at least one isolated nucleic acid comprises in vitro transcribed RNA.

21. The method of claim 17, wherein the at least one isolated nucleic acid comprises nucleoside-modified RNA.

22. The method of claim 17, wherein the at least one isolated nucleic acid comprises pseudouridine.

23. The method of claim 17, wherein the composition comprises an isolated nucleic acid encoding an RNase, an isolated nucleic acid encoding a DNase, an isolated nucleic acid encoding an ENTPD, and an isolated nucleic acid encoding NT5E.

24. The method of claim 17, wherein the composition further comprises an isolated nucleic acid encoding an inhibitor of HMGB1.

25. The method of claim 17, wherein the composition further comprises an isolated nucleic acid encoding an inhibitor of peroxiredoxins.

26. The method of claim 17 wherein the composition further comprises a cell debris-inhibiting or -catabolizing peptide.

27. The method of claim 17, wherein the composition is administered by a delivery method selected from the group consisting of intravenous delivery, intranasal delivery, and intracerebroventricular delivery.
